(19) United States
(12) Patent Application Publication
(10) Pub. No.: US 2008/0064632 A1
(43) Pub. Date: Mar. 13, 2008

(54) Combination Therapy for the Treatment of Obesity

(76) Inventors: John M. Amatruda, New York, NJ (US); Paul Daruwala, Branchburg, NJ (US); Ngozi E. Erondu, Englishtown, NJ (US); Douglas J. MacNeil, Westfield, NJ (US); David E. Moller, Bedminster, NJ (US); Su Qian, Princeton, NJ (US)

Correspondence Address: MERCK AND CO., INC, P O BOX 2000, RAHWAY, NJ, 07065-0907, US

(21) Appl. No.: 11/661,960

(22) PCT Filed: Sep. 22, 2005
(86) PCT No.: PCT/US05/34096

§ 371 Date: Mar. 2, 2007

Related U.S. Application Data
(60) Provisional application No. 60/612,657, filed on Sep. 24, 2004.

(51) Int. Cl. A61K 38/17; A61P 3/04

(52) U.S. Cl. 514/12

(57) ABSTRACT

The present invention relates to compositions comprising PYY, PYY₃₋₃₆, or a PYY agonist, and an anti-obesity agent, useful for the treatment and prevention of obesity and obesity-related disorders. The present invention further relates to methods of treating or preventing obesity and obesity-related disorder in a subject in need thereof by administering a composition of the present invention. The present invention further provides for pharmaceutical compositions, medicaments, and kits useful in carrying out these methods.

### BACKGROUND OF THE INVENTION

Obesity, which can be defined as a body weight more than 20% above the ideal body weight, is a major health concern in Western societies. It is estimated that about 97 million adults in the United States are overweight or obese. Obesity is the result of a positive energy balance, as a consequence of increased ratio of caloric intake to energy expenditure. The molecular factors regulating food intake and body weight balance are incompletely understood [B. Staels et al., J. Biol. Chem. 270(27), 15958 (1995); F. Lonnquist et al., Nature Medicine 1(9), 950 (1995)]. Although the genetic and/or environmental factors leading to obesity are poorly understood, several genetic factors have been identified.

Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometirial, breast, prostate and colon cancer; osteoarthritis; respiratory complications, such as obstructive sleep apnea; cholelithiasis; gallstones; arteriosclerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Obesity is often treated by encouraging patients to lose weight by reducing their food intake or by increasing their exercise level and therefore increasing their energy output. A sustained weight loss of 5% to 10% of body weight has been shown to improve the co-morbidities associated with obesity, such as diabetes and hypertension, and can lead to improvement of obesity-related conditions such as osteoarthritis, sleep apnea and pulmonary and cardiac dysfunction.

Weight loss drugs that are currently used in monotherapy for the treatment of obesity have limited efficacy and significant side effects. Studies of the weight loss medications orlistat (Davidson, M. H. et al. (1999) JAMA 281: 235-42), dexfenfluramine (Guy Grand, B. et al. (1989) Lancet 2: 114-2-5), sibutramine (Bray, G. A. et al. (1999) Obes. Res. &: 189-98) and phentermine (Douglas, A. et al. (1983) Int. J. Obes. 7: 591-5) have demonstrated a limited weight loss of about 5%-10% of body weight for drug compared to placebo. In particular, both sibutramine and orlistat reduce body weight less than 10% over a 6 month or a 1 year period. Preclinical studies have also found that most agents, such as sibutramine, fenfluramine, NPY5 antagonists, CB-1 inverse agonists and Mc4r agonists, potently inhibit food intake and decrease body weight initially. However, during chronic treatment periods of greater than 10 days the efficacy of these agents decreases yielding no more than 10% body weight loss compared to control. Obese humans can easily mass over 150 kg and would, therefore, need to lose more than 50% of their body mass to return to a normal body mass. For these patients, single agents are likely to have minimal therapeutic utility. The side effects of these drugs and anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

While single agents may be efficacious for the treatment of obesity in certain patients, due to the polygenic nature of obesity etiology, it is predicted that no single agent will be efficacious for the vast majority of obese patients. Current therapies do not show continued improvement in weight loss after 6 months to 1 year. Combination therapy targeting simultaneously independent pathways is more likely to efficacious in achieving greater sustained weight loss. Additionally, combination therapy is more likely to achieve the desired medical benefits without the trial and error involved in prescribing each agent individually during primary care.

Commercially available combination therapies, which include phentermine as one of the components, have lead to mixed results. Phentermine was prescribed with fenfluramine (Pondimin®) or dexfenfluramine (Redux®) as a combination therapy known as fenphen, which was withdrawn from the market in 1997 based on studies suggesting that the drugs cause damage to the mitral valve of the heart and pulmonary hypertension. Additionally, both fenfluramine and phentamine (phentermine) work through the same mechanism, namely the serotonin and norepinephrine pathway.

Due to the side effects and limited efficacy of the anti-obesity drugs currently available for mono- and combination therapy, there is a need for a combination weight loss treatment with enhanced efficacy, sustained efficacy, and fewer undesirable side effects. The instant invention addresses this problem by providing a combination therapy comprised of PYY (SEQ ID No. 1, FIG. 7), PYY₃₋₃₆ (SEQ ID No. 2, FIG. 8), or a PYY agonist and a second anti-obesity agent useful in the treatment and prevention of obesity and obesity-related disorders.

It has now been found that the combination of PYY₃₋₃₆ and an anti-obesity agent, that decreases appetite or food intake, increases the metabolic rate or inhibits nutrient absorption, is advantageous in the treatment of obesity over treatment with either PYY₃₋₃₆ or the anti-obesity agent alone. The compositions of the present invention are more effective than currently available mono- and combination therapies based on the mode of action of PYY, PYY₃₋₃₆, or a PYY agonist, and the second anti-obesity agent in these compositions. Additionally, treatment with the compositions of the present invention allow the use of the maximum efficacious dose of PYY₃₋₃₆, which has no significant side effects, and a sub-clinical dose of a second anti-obesity agent, with known side effects, resulting in effective treatment with fewer side effects than current monotherapies.

It is an object of the present invention to identify compositions comprising PYY, PYY₃₋₃₆, or a PYY agonist, and an anti-obesity agent useful for the treatment of obesity and obesity-related diseases. It is another object of the invention to identify methods of treating obesity. It is yet another object of the invention to identify methods of preventing obesity. It is a further object of the present invention to provide pharmaceutical compositions comprising PYY, PYY₃₋₃₆ or a PYY agonist, and a second anti-obesity agent. It is yet a further object of the present invention to provide a method of manufacture of a medicament useful in the treatment of obesity.

### SUMMARY OF THE INVENTION

The present invention provides compositions comprising PYY, PYY₃₋₃₆ or a PYY agonist, and an anti-obesity agent useful in the treatment or prevention of obesity, and obesity-related disorders.

The present invention is further concerned with compositions comprising a PYY, PYY₃₋₃₆ or a PYY agonist, and a second anti-obesity agent selected from the group consisting of: 5HT transporter inhibitor; NE transporter inhibitor; CB-1 antagonist/inverse agonist; ghrelin antagonist; H3 antagonist/inverse agonist; MCH1R antagonist; MCH2R agonist/antagonist; MC3R agonist; MC4R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor, CCK-A agonist; CNTF; CNTF agonist/modulator; CNTF derivative; Cox-2 inhibitor; DP-IV inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; GLP-1 agonist; ACC1 inhibitor; dual ACC 1 and 2 inhibitor; stearoyl CoA denaturase inhibitor; GLP-1; GLP-1 analog; neuromedin U, neuromedin U receptor agonist; oxyntomodulin; oxyntomodulin analog; dopamine/serotonin dual reuptake inhibtor; serotonin/norepinephrine dual reuptake inhibtor; dopamine/norepinephrine dual reuptake inhibtor; 5HT/dopamine/norepinephrine triple reuptake inhibtor; amfepramone (diethylpropion), a minorex; amphechloral; amphetamine; axokine; benzphetamine; bupropion; chlorphentermine; clobenzorex; cloforex; clominorex; clortermine; cyclexedrine; dexfenfluramine; dextroamphetamine; diphemethoxidine, N-ethylamphetamine; fenbutrazate; fenfluramine, fenisorex; fenproporex; fludorex; fluminorex; fluoxetine; furfurylmethylamphetamine; levamfetamine; levophacetoperane; mazindol; mefenorex; metamfepramone; methamphetamine; nalmefene; norpseudoephedrine; pentorex; phendimetrazine; phenmetrazine; phentermine; phenylpropanolamine; phytopharm compound 57; picilorex; zonisamide; metformin; radafaxine HCl; and topiramate. The compositions of the present invention are useful in the treatment or prevention of the following obesity related disorders: overeating; bulimia; hypertension; diabetes, elevated plasma insulin concentrations; insulin resistance; dyslipidemias; hyperlipidemia; endometrial, breast, prostate and colon cancer; osteoarthritis; obstructive sleep apnea; cholelithiasis; gallstones; abnormal heart rhythms; heart arrythmias; myocardial infarction; congestive heart failure; coronary heart disease; sudden death; stroke; polycystic ovarian disease; craniopharyngioma; the Prader-Willi Syndrome; Frohlich's syndrome; GH-deficient subjects; normal variant short stature; Turner's syndrome; metabolic syndrome; and other pathological conditions showing reduced metabolic activity or a decrease in resting energy expenditure as a percentage of total fat-free mass, e.g, children with acute lymphoblastic leukemia.

The present invention also relates to the treatment of these conditions, and the use of the compositions of the present invention for manufacture of a medicament useful for treating these conditions.

The invention also relates to pharmaceutical compositions comprising PYY, PYY₃₋₃₆ or a PYY agonist, and a second anti-obesity agent, as active ingredients.

The present invention also relates to the use of PYY, PYY₃₋₃₆ or a PYY agonist, and a second anti-obesity agent for the manufacture of a medicament for the treatment of obesity which comprises an effective amount of PYY, PYY₃₋₃₆ or a PYY agonist, and an effective amount of anti-obesity agent, together or separately.

The present invention is also concerned with a product containing a PYY, PYY₃₋₃₆ or a PYY agonist, and a second anti-obesity agent as a combined preparation for simultaneous, separate or sequential use in obesity.

The present invention also relates to the treatment of obesity with a combination of a PYY, PYY₃₋₃₆ or a PYY agonist, and an anti-obesity agent which may be administered separately, the invention also relates to combining separate pharmaceutical combinations into a kit form.

### BRIEF DESCRIPTION OF THE DRAWING

FIG. 1. Shows the inhibition of 4 hour food intake in diet induced obese (DIO) mice induced by PYY₃₋₃₆ (0.03 to 5 mg/kg; I.P.), compared to a control group given saline.

FIG. 2. Shows the inhibition of 16 hour food intake in diet induced obese (DIO) mice induced by PYY₃₋₃₆ (0.03 to 5 mg/kg; I.P.), compared to a control group given saline.

FIG. 3. Shows inhibition of overnight (16 hour) body weight gain in diet induced obese (DIO) mice following administration of PYY₃₋₃₆ (0.03 to 5 mg/kg; I.P.), as compared to a control group given saline alone.

FIG. 4. Shows an increased inhibition of food intake in diet induced obese (DIO) mice following administration of a combination of PYY₃₋₃₆ (1 mg/kg; I.P.) and cannabinoid 1 receptor inverse agonist AM 251 (1 mg/kg; P.O.), as compared to control groups given saline alone, PYY₃₋₆ alone, and AM 251 alone.

FIG. 5. Shows a greater decrease in overnight body weight gain in diet induced obese (DIO) mice following administration of a combination of PYY₃₋₃₆ (1 mg/kg; I.P.) and cannabinoid 1 receptor inverse agonist AM 251 (1 mg/kg, P.O.), as compared to control groups given saline alone, PYY₃₋₆ alone, and AM 251 alone.

FIG. 6. Shows a greater decrease in 4 hour food intake in diet induced obese (DIO) mice following administration of a combination of PYY₃₋₃₆ (1 mg/kg; I.P.) and melanocortin 4 receptor agonist, Compound A (20 mg/kg; P.O.), as compared to control groups given saline alone, PYY₃₋₃₆ alone, and Compound A alone.

FIG. 7. Shows the sequence listing of human peptide YY [SEQ ID NO.: 1].

FIG. 8. Shows the sequence listing of human peptide YY 3-36 [SEQ ID NO.: 2].

FIG. 9. Shows a greater decrease in 2 week food intake in diet induced obese (DIO) mice following administration of a combination of PYY₃₋₃₆ (1 mg/kg; SC) and NPY5 antagonist, Compound B (30 mg/kg; oral), as compared to control groups given saline alone, PYY₃₋₃₆ alone, and Compound B alone.

FIG. 10. Shows a greater decrease in 2 week body weight gain in diet induced obese (DIO) mice following administration of a combination of PYY₃₋₃₆ (1 mg/kg; SC) and NPY5 antagonist Compound B (30 mg/kg, oral.), as compared to control groups given saline alone, PYY₃₋₆ alone, and Compound B alone.

### DETAILED DESCRIPTION OF THE INVENTION

Peptide YY (PYY) is a 36 amino acid residue peptide amide isolated originally from porcine intestine and localized in the endocrine cells of the gastrointestinal tract and the pancreas (Tatemotu et al., Proc. Natl. Acad. Sci. 79: 2514, (1982)). Peptide YY (PYY) is a member of the neuropeptide Y (NPY) family which also includes neuropeptide Y (NPY) and pancreatic polypeptide (PP). PYY has been shown to stimulate food and water intake after central administration (Levine et al, Brain Res 341: 200-203 (1985); Corp, Melville et al. Am J Physiol 259: R317-23, (1990); U.S. 2002/0141985). However, peripherally injected peptide YY has been shown to produce weight loss in mice (Morley, J. Neuropsychobiology 21: 22-30 (1989)). Studies have also shown that PYY binds to the neuropeptide Y2 receptor and thereby causes a decrease in the ingestion of carbohydrate, protein and meal size (Leibowitz, S. F. et al. Peptides, 12. 1251-1260 (1991)). PYY is converted by dipeptidyl peptidase IV into the metabolite PYY₃₋₃₆, which is a more highly selective agonist for the neuropeptide Y2 receptor (NY2R) (Santos Medeiros, et al., Endocrinology, Vol. 134, No. 5, 2088-2094 (1994); Eberlein, et al. Peptides 10: 797-803, (1989); Eysselein, et al., Peptides 11: 111-116, (1990); Grandt, et al. Regul Pept 51: 151-9, (1994)).

The gut peptide PYY₃₋₃₆ is also produced and released into the circulation from the gastrointestinal tract after meal consumption in proportion to the calorie content of a meal. The anorectic effect of PYY₃₋₃₆ is abolished in the neuropeptide Y2 receptor deficient mice, suggesting that the effect is mediated through NPY2R signaling (Batterham et al., Nature, 418, 8, 650-654 (2002). PYY₃₋₃₆ injected into arcuate nucleus suppresses food intake, suggesting action on arcuate nucleus Y2 receptors (Batterham et al., Nature, 418, 8, 650-654 (2002). Recently it has been reported that infusion of normal postprandial concentrations of PYY₃₋₆ significantly reduces appetite and food intake in humans (Batterham et al., Nature, 418, 8, 650-654 (2002); Batterham et al., N. Engl. J. Med., 349, 10, 941-948 (2003)). However, several subsequent studies found that PYY₃₋₃₆ does not decrease food intake in rodents (Tschop, M. et al., Nature, 430, 6996, Jul. 8, 2004).

Mechanisms of appetite control are complex and involve multiple regulatory circuitries. Monotherapy targeting one pathway is subject to compensatory rebound over time, thus combination therapy offers more greater efficacy in the treatment of obesity than monotherapy.

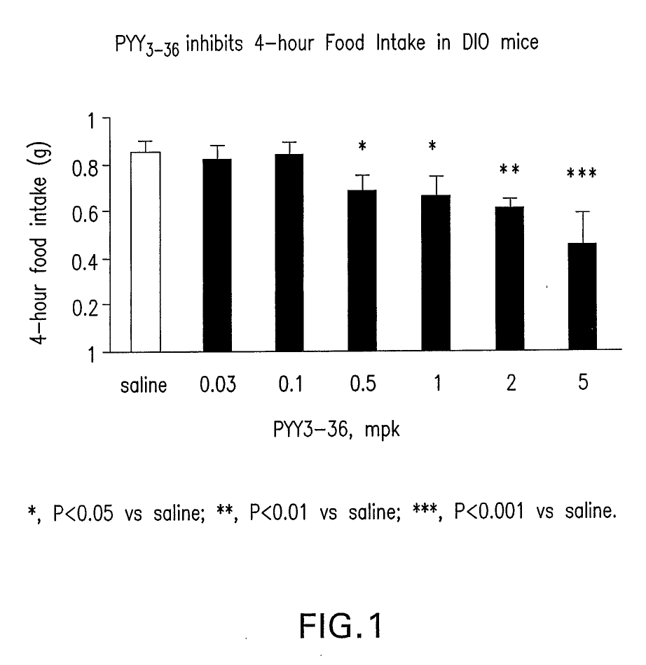

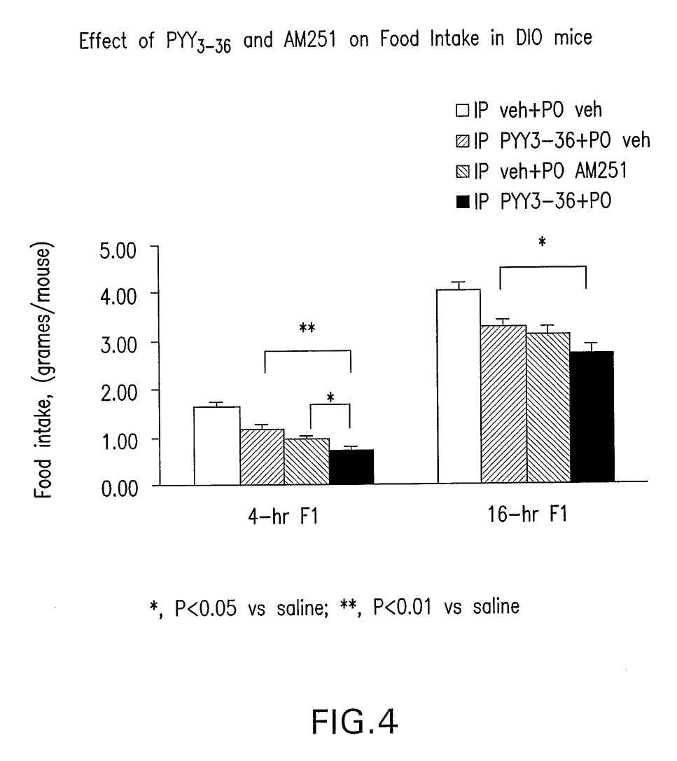

One approach to obesity therapy is reducing food intake (FI). In the present invention, it was surprisingly found that PYY₃₋₃₆ decreases 4 and 16 hour food consumption and decreases overnight body weight gain in diet induced obese (DIO) mice (See FIGS. 1, 2 and 3). In the studies of the present invention, it was also found that co-administration of PYY₃₋₃₆ with another anti-obesity agent had greater anorectic efficacy than the administration of either compound alone. Specifically, it was found that co-administration of PYY₃₋₃₆ and cannabinoid receptor-1 (CB 1) inverse agonist AM251 resulted in greater food intake inhibition in diet-induced-obese (DIO) mice than either compound administered alone (See Table 1, FIG. 4).

TABLE 1
Effects of Co-administration of PYY₃₋₃₆ and AM 251

| Compound | 4-hour FI | 16-hour FI |
|---|---|---|
| PYY3-36 @ 1 mpk | −30% | −19% |
| AM251 @ 1 mpk | −42% | −23% |
| PYY3-36 + AM251 | −57% | −34% |

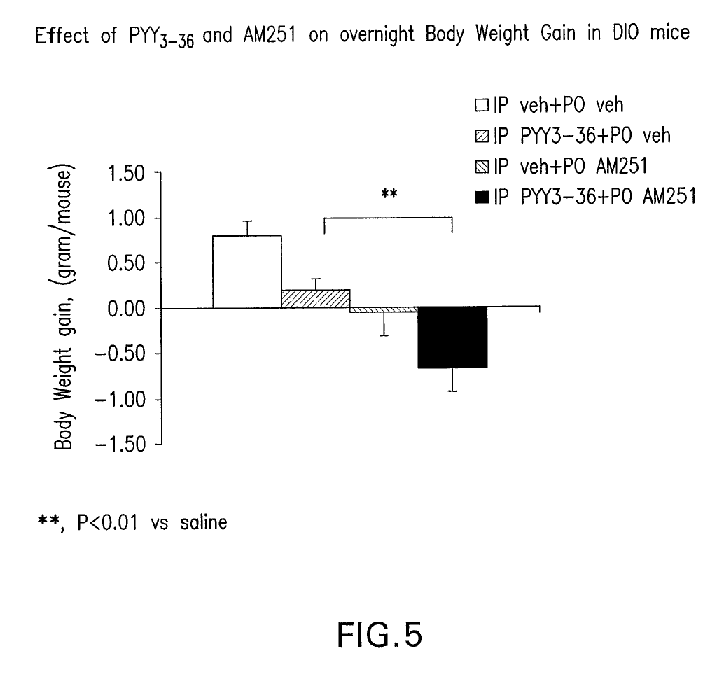

It was also found that co-administration of PYY₃₋₃₆ and cannabinoid receptor-1 (CB 1) inverse agonist AM251 resulted in a greater decrease in bodyweight gain in diet-induced-obese (DIO) mice than either compound administered alone (See FIG. 5).

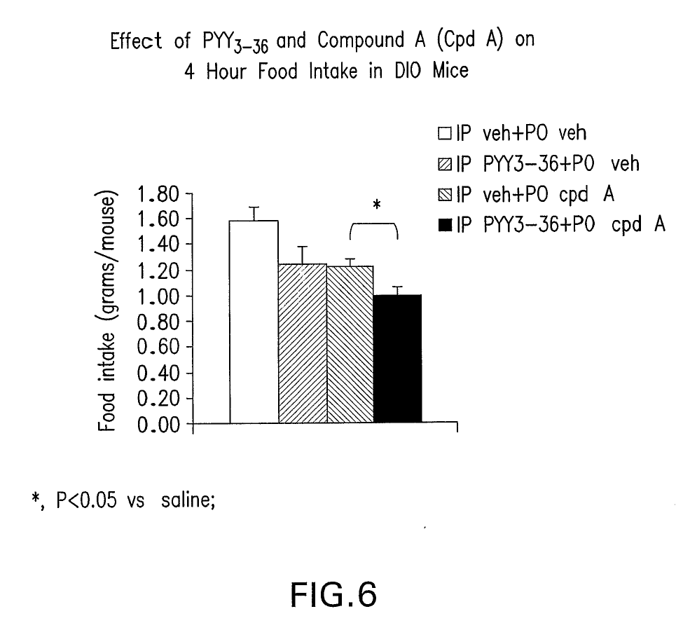

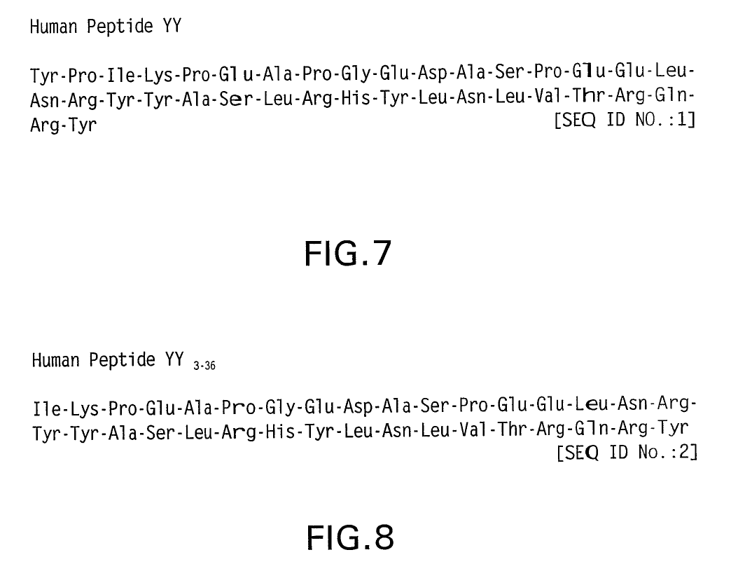

Additionally, the studies of the present invention show that co-administration of PYY₃₋₆ with a MC4R agonist, Compound A, resulted in greater food intake inhibition than treatment with either individual compound alone in a 4-hour food intake study in DIO C57B6 mice (See FIG. 6).

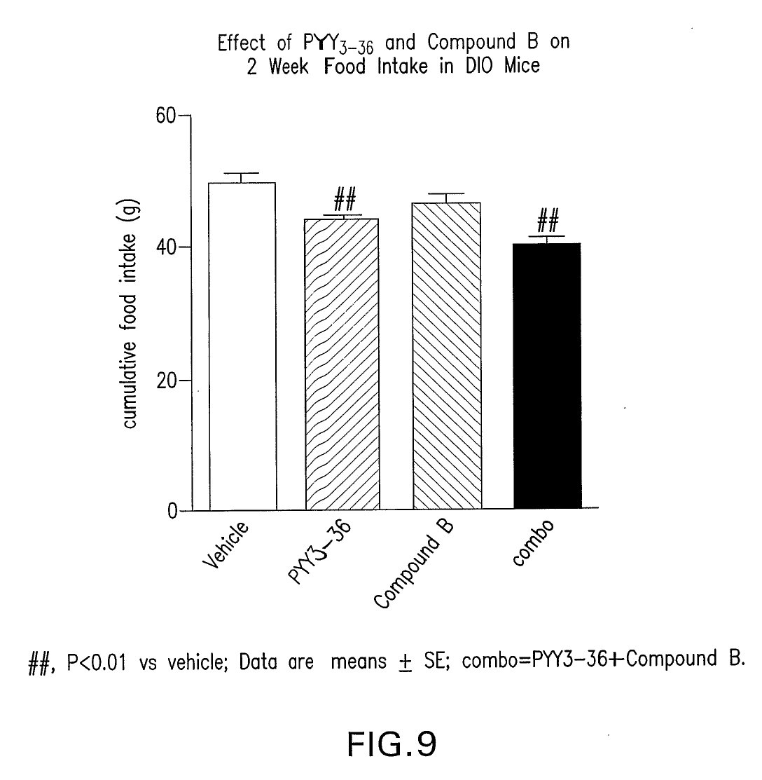

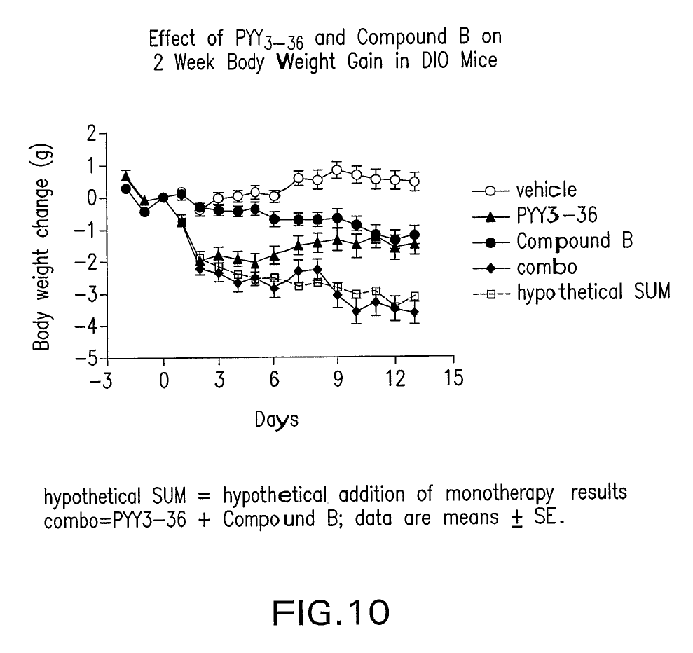

Finally, the studies of the present invention show that co-administration of PYY₃₋₆ with a NPY5 antagonist, Compound B, resulted in a greater decrease in bodyweight gain in diet-induced-obese (DIO) mice than either compound administered alone (see FIG. 9), and resulted in greater food intake inhibition than treatment with either individual compound alone in a 2 week food intake study in DIO mice (see FIG. 10). Additionally, the co-administration of PYY₃₋₆ with a NPY5 antagonist, Compound B, resulted in a greater decrease in bodyweight gain in diet-induced-obese (DIO) mice than the hypothetical sum of the monotherapy results (see FIG. 10). Further studies also showed that there vas a greater reduction in fat mass for the combination of PYY₃₋₃₆ and NPY5 antagonist, Compound B, than for each drug alone.

These studies show that treatment with PYY₃₋₃₆ and a second anti-obesity agent results in greater food intake inhibition, greater weight loss, and greater resistance to weight gain than treatment with either PYY₃₋₃₆ or the second anti-obesity agent alone. These data indicate that PYY₃₋₃₆ in combination with a CB1 inverse agonist/antagonist or a MC4R agonist would be more efficacious in reducing food intake, reducing body weight, reducing body weight gain, and in treating obesity than treatment with either single agent alone. Based on the findings of increased food inhibition and decreased body weight gain resulting from the combination of PYY₃₋₃₆ and these two mechanistically distinct anti-obesity agents, AM 251 (a CB-1 antagonist/inverse agonist) and Compound A (a melanocortin 4 receptor agonist), other anti-obesity agents will also be effective when combined with PYY₃₋₃₆.

The combination of PYY₃₋₃₆, PYY or a PYY agonist and a second anti-obesity agent is useful for reducing food intake. The combination of PYY₃₋₃₆, PYY or a PYY agonist and a second anti-obesity agent is useful for reducing body weight. The combination of PYY₃₋₃₆, PYY or a PYY agonist and a second anti-obesity agent is useful for treating obesity. The combination of PYY₃₋₃₆, PYY or a PYY agonist and a second anti-obesity agent is useful for prolonging resistance to weight gain and weight regain. Additionally, the combination of PYY₃₋₃₆, PYY or a PYY agonist and a second anti-obesity agent is useful for maintaining weight loss, including weight loss due to any cause, including but not limited to diet, drug therapy and exercise. Furthermore, the co-administration of PYY₃₋₃₆ and a second obesity agent would result in greater food intake inhibition, greater body weight gain inhibition and greater weight loss than either agent administered alone. Likewise, the co-administration of PYY and a second obesity agent would result in greater food intake inhibition, greater body weight gain inhibition and greater weight loss than either agent administered alone due to the conversion of PYY to PYY₃₋₃₆ in vivo. Additionally, the co-administration of a PYY agonist that binds to a NPY2 receptor and induces the same physiological responses as PYY or PYY₃₋₃₆, and a second anti-obesity would result in greater food intake inhibition, greater body weight gain inhibition and greater weight loss than either agent administered alone.

The present invention provides compositions comprising PYY, PYY₃₋₃₆ or a PYY agonist, and a second anti-obesity agent useful in the treatment or prevention of obesity and obesity-related disorders. The present invention further provides for co-administration of PYY, PYY₃₋₃₆ or a PYY agonist, and a second anti-obesity agent to reduce food intake, reduce body weight, reduce body weight regain, to slow body weight gain, to treat or prevent obesity and to treat or prevent obesity related disorders.

The methods and compositions of the present invention comprise PYY, PYY₃₋₃₆, or a PYY agonist.

The amino acid sequences of human PYY and human PYY₃₋₃₆ are as follows:

human PYY:
YPIKPEAPGEDASPEELNRYYASLRHYLNLVTRQRY
human PYY₃₋₃₆:
IKPEAPGEDASPEELNRYYASLRHYLNLVTRQRY

The term “PYY agonist” as used herein, refers to a small molecule or a peptide, for example, a PYY fragment, a PYY analog, a PYY functional analog, or a PYY derivative, that binds to the neuropeptide Y2 receptor and produces a physiological response similar to PYY or PYY₃₋₆ leading to a reduction in food intake or weight loss. For example, a PYY agonist is a compound (1) having activity in food intake reduction or weight loss assays, and (2) which binds specifically to the neuropeptide Y 2 receptor in a NPY2 receptor assay or in a competitive binding assay with labeled PYY or PYY₃₋₃₆; but is not pancreatic polypeptide (PP).

The term “PYY agonist” as used herein may be a fusion protein containing the PYY peptide sequence or may be formed by modifying the PYY peptide's natural amino acid sequence or modifying the N-terminal amino and/or the C-terminal carboxyl group, and include salts formed with acids and/or bases, particularly physiologically acceptable inorganic and organic acids and bases. Preferred modifications are those which provide a more stable, active peptide which will be less prone to enzymatic degradation in vivo.

PYY agonists include, but are not limited to, PYY analogs prepared to emulate and preferably enhance the duration of effect, biological activity, and selectivity of the endogenous ligands PYY and PYY₃₋₃₆. PYY analogs may be synthetic or naturally occurring PYY analogs. Synthetic analogs of PYY refer to polypeptides with natural or synthetic amino acid substitutions replacing the endogenous ligand PYY, for example, [Pro 34]PYY of SEQ ID No. 1 and [D-Trp32]PYY of SEQ ID No. 1. Many of these analogs are derived from biologically active peptide fragments of PYY (e.g., PYY₂₂₋₃₆ of SEQ ID No 1, and PYY₂₅₋₃₆ of SEQ ID No. 1). PYY agonists and PYY analogs useful in the present invention include, but are not limited to, those disclosed in U.S. Pat. No. 5,026,685, U.S. Pat. No. 5,604,203, U.S. Pat. No. 5,574,010, U.S. Pat. No. 5,696,093, U.S. Pat. No. 5,936,092, U.S. Pat. No. 6,046,162, U.S. Pat. No. 6,046,167, U.S. Pat. No. 6,093,692, U.S. Pat. No. 6,225,445, WO 03/026591, and WO 2004/066966, which are incorporated herein by reference. PYY agonists also include PYY derivatives, such as those disclosed in U.S. Pat. No. 4,002,531, U.S. Pat. No. 4,179,337, U.S. Pat. No. 5,122,614, U.S. Pat. No. 5,349,052, U.S. Pat. No. 5,552,520, U.S. Pat. No. 6,127,355, WO 95/06058, WO 98/32466, and WO 03/026591, which are incorporated herein by reference.

PYY agonists include PYY functional analogs, including, but not limited to, BIM-43073D, BIM-43004C (Olitvak, D. A. et al., Dig. Dis. Sci. 44(3):64348 (1999)), and those disclosed in Liu, C D. et al., Surg. Res. 59(1):80-84 (1995) and U.S. Pat. No. 5,604,203, which is incorporated herein by reference.

PYY agonists also include NPY 2 receptor agonists with activity in food intake reduction and weight loss. Neuropeptide Y2 (NPY2) receptor agonists useful in the present invention, include, but are not limited to compounds such as PYY3-36 as described in Batterham, et al., Nature. 418: 650-654 (2003), NPY3-36, and other Y2 agonists such as N acetyl [Leu(28, 31)] NPY 24-36 (White-Smith and Potter, Neuropeptides 33: 526-33 (1999)), TASP-V (Malis et al., Br. J. Pharmacol. 126: 989-96 (1999)), cyclo-(28/32)-Ac-[Lys28-Glu32]-(25-36)-pNPY (Cabrele and Beck-Sickinger, J-Pept-Sci. 6:97-122 (2000)).

PYY agonists include, but are not limited to: PYY ligands from different species, peptide PYY agonists (including PYY₃₋₆ variations, NPY variations, NPY agonists, NPY analogs, PP variations), conservative amino acid substitutions (including single and double point mutations of PYY and PYY₃₋₃₆ fragments); neurophilic Y Y2 receptor specific peptides, PYY modifications and PYY agonist modifications, modified derivatives of PYY and PYY agonists, and drug carrier conjugates including, but not limited to, those disclosed in PCT Application No. WO 03/026591.

Additional PYY agonists which can be used to practice the therapeutic method of the present invention include, but are not limited to, those specifically recited in the publications set forth below: U.S. Pat. Nos. 5,604,203 and 5,574,010, which are incorporated herein by reference; Balasubramaniam, et al., Peptide Research 1: 32 (1988); Japanese Patent Application 2,225, 497 (1990); Balasubramaniam, et al., Peptides 14: 1011, 1993; Grandt, et at., Reg. Peptides 51, 151, (1994); and PCT International Applications WO 94/03380, WO 94/22467, and WO 98/20885.

One of ordinary skill in the art can readily identify PYY agonists useful in the compositions and methods of the present invention using the binding assays and functional assays described Gehlert, D. R. et al., Mol-Pharmacol. 49: 224-8, (1996); and Nakamura, et al., Biochim.Biophys.Acta. 1284: 134-7 (1998). PYY agonists which are useful in the present invention generally have an IC₅₀ less than 1 μM in the NPY Y2 binding assay described in Gehlert, D. R. et al., Mol-Pharmacol. 49: 224-8, (1996). NPY2 agonists which are preferred in the present invention generally have an IC₅₀ less than 100 nM in the NPY Y2 binding assay, more preferably an IC₅₀ of less than 10 nM, most preferably an IC₅₀ less than 1 nM.

The methods and compositions of the present invention comprise an anti-obesity agent. The anti-obesity agents useful in the compositions of the present invention include: 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5H1T6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; and serotonin reuptake inhibitors; and pharmaceutically acceptable salts and esters thereof.

In one embodiment of the present invention, the composition is comprises of PYY, or a pharmaceutically acceptable salt or ester thereof. In another embodiment of the present invention, the composition is comprised of PYY₃₋₃₆, or a pharmaceutically acceptable salt or ester thereof. In another embodiment of the present invention, the composition is comprised of a PYY agonist, or a pharmaceutically acceptable salt or ester thereof.

In another embodiment of the present invention, the anti-obesity agent is selected from the group consisting of: NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; orexin antagonist; BRS3 agonist; 11β HSD-1 inhibitor; CNTF; CNTF agonist/modulator; CNTF derivative; Cox-2 inhibitor; GHS agonist; monoamine reuptake inhibitor; UCP-1, 2, and 3 activator; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; and dicarboxylate transporter inhibitor; and pharmaceutically acceptable salts and esters thereof.

In one class this embodiment of the present invention, the anti-obesity agent is selected from the group consisting of: aminorex; amphechloral; amphetamine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; norpseudoephedrine; orlistat; pentorex; phendimetrazine; phenmetrazine; picilorex; phytopharm 57; zonisamide; and topiramate; and pharmaceutically acceptable salts and esters thereof.

In another class of this embodiment, the anti-obesity agent is selected from the group consisting of: acyl-estrogen; opioid antagonist; monoamine reuptake inhibitor; lipase inhibitor; leptin; CNTF; CNTF derivatives; and NPY5 antagonist; and pharmaceutically acceptable salts and esters thereof.

In another class, the acyl-estrogen is selected from oleoyl-estrone, and the pharmaceutically acceptable salts thereof.

In another class, the monoamine reuptake inhibitor is selected from sibutramine, and the pharmaceutically acceptable salts thereof.

In another class, the CNTF derivative is selected from axokine, and the pharmaceutically acceptable salts thereof.

In another class, the lipase inhibitor is selected from orlistat, and the pharmaceutically acce

In another class, the anti-obesity agent is selected from leptin, and the pharmaceutically acceptable salts thereof.

In another class, the opioid antagonist is selected from nalmefene, and the pharmaceutically acceptable salts thereof.

In another class, the anti-obesity agent is selected from the group consisting of: dexfenfluramine, fenfluramine, and phentermine, and pharmaceutically acceptable salts thereof.

In another class, the anti-obesity agent is Phytopharm compound 57 (CP 644, 673).

In another class, the anti-obesity agent is selected from the group consisting of zonisamide, and pharmaceutically acceptable salts and esters thereof. In another class, the anti-obesity agent is selected from topiramate, and the pharmaceutically acceptable salts thereof.

In another class, the anti-obesity agent is selected from metformin, and the pharmaceutically acceptable salts thereof.

In another embodiment of the present invention, the anti-obesity agent is selected from a Mc4r agonist, and pharmaceutically acceptable salts and esters thereof.

In one class of this embodiment, the Mc4r agonist is compound A. Compound A has the following structure: 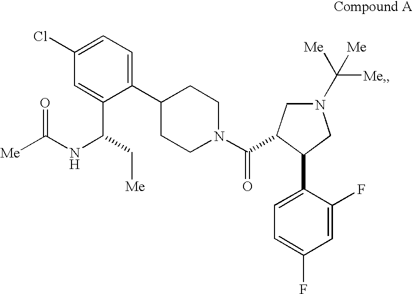
and pharmaceutically acceptable salts, esters and tautomers thereof. Compound A, and its preparation are disclosed in WO 02/068388.

In a subclass of this class, the composition comprises PYY3-36, or a salt or ester thereof; and a Mc4r agonist selected from the group consisting of: 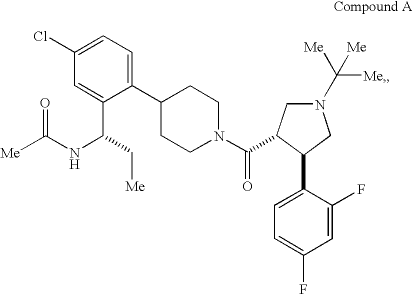
and pharmaceutically acceptable salts and esters thereof.

In another class of this embodiment, the anti-obesity agent is a Mc4r agonist selected from the group consisting of:
(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide,
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide,
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide,
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide,
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide,
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide,
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine,
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine,
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine,
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine,
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine,
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine,
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide,
(15) N-{1-[2-(1-{[(3S,4R)-1-tert-butyl-4-(2,4-difluorophenyl)pyrrolidin-3-yl]carbonyl}piperidin-4-yl)-5-chlorophenyl]1-nmethylethyl}acetamide,
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide,
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide,
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide,
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea,
(20) Methyl-2-[2-(1-{[(3S,4R)-1-tert-butyl-4-(2,4-difluoro-phenyl)pyrrolidin-3-yl]carbonyl}piperidin-4-yl)-5-chlorophenyl]-2-methylpropanoate,
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide,
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea,
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide,
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide,
(25) N-{(1S)-1-[2-(1-{[(3S,4R)-1-tert-butyl-4-(2,4-difluoro-phenyl)pyrrolidin-3-yl]carbonyl}piperidin-4-yl)-5-fluorophenyl]ethyl}acetamide,
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide, and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide,
   and pharmaceutically acceptable salts thereof.

In a sub-class of this class, the anti-obesity agent is a Mc4r agonist selected from the group consisting of:
(1) N-{(1S)-1-[2-(1-{[(3S,4R)-1-tert-butyl-4-(2,4-difluoro-phenyl)pyrrolidin-3-yl]carbonyl}piperidin-4-yl)-5-fluorophenyl]ethyl}acetamide,
(2) N-{(1S)-1-[2-(1-{[(3S,4R)-1-tert-butyl-4-(2,4-difluoro-phenyl)pyrrolidin-3-yl]carbonyl}piperidin-4-yl)-5-chlorophenyl]propyl}acetamide,
(3) N-{(1S)-1-[2-(1-{[(3S,4R)-1-tert-butyl-4-(2,4-difluoro-phenyl)pyrrolidin-3-yl]carbonyl}piperidin-4-yl)-5-chlorophenyl]ethyl}acetamide,
(4) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide,
(5) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide, and
(6) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine,
   and pharmaceutically acceptable salts thereof.

In another embodiment of the present invention, the anti-obesity agent is a CB-1 antagonist/inverse agonist, or a pharmaceutically acceptable salt or ester thereof.

In a class of this embodiment, the CB-1 antagonist/inverse agonist is selected from rimonabant, and the pharmaceutically acceptable salts thereof. In a subclass of this class, the composition comprises PYY3-36, or a pharmaceutically acceptable salt or ester thereof; and rimonabant, or a pharmaceutically acceptable salt or ester thereof.

In another class of this embodiment, the CB-1 antagonist/inverse agonist is selected from AM 251, and the pharmaceutically acceptable salts thereof. In a subclass of this class, the composition comprises PYY3-36, or a pharmaceutically acceptable salt or ester thereof; and AM 251, or a pharmaceutically acceptable salt or ester thereof.

AM-251, also known as SR 141716A, has the following structure: 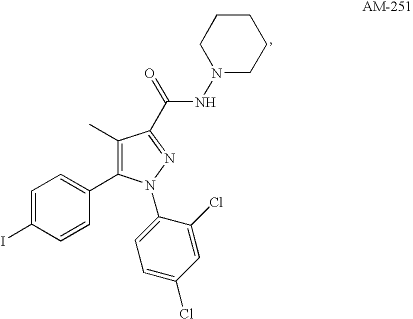
and pharmaceutically acceptable salts, esters and tautomers thereof.

In a class of this embodiment, the composition comprises PYY3-36, or a salt or ester thereof; and AM251, or a salt or ester thereof.

In another embodiment of the present invention, the anti-obesity agent is selected from a NPY5 antagonist, and pharmaceutically acceptable salts and esters thereof.

In a class of this embodiment of the present invention, the anti-obesity agent is a NPY5 antagonist of structural Formula I: 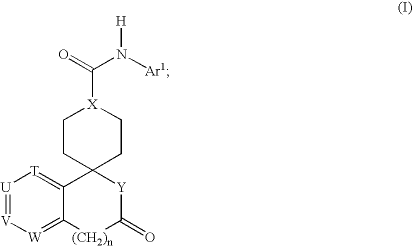
and pharmaceutically acceptable salts, esters and tautomers thereof, wherein Ar¹ is selected from the group consisting of:

(1) aryl, and

(2) heteroaryl,

wherein the aryl and heteroaryl groups are unsubstituted or optionally substituted with a substituent selected from the group consisting of:

(a) halogen,

(b) nitro,

(c) lower alkyl,

(d) halo(lower)alkyl,

(e) hydroxy(lower)alkyl,

(f) cyclo(lower)alkyl,

(g) lower alkenyl,

(h) lower alkoxy,

(i) halo(lower)alkoxy,

(j) lower alkylthio,

(k) carboxyl,

(l) lower alkanoyl,

(m) lower alkoxycarbonyl,

(n) lower alkylene optionally substituted with oxo, and

(o) -Q-Ar²;

Ar² is selected from the group consisting of

(1) aryl, and

(2) heteroaryl,

wherein aryl and heteroaryl are unsubstituted or optionally substituted with a substituent selected from the group consisting of:

(a) halogen,

(b) cyano,

(c) lower alkyl,

(d) halo(lower)alkyl,

(e) hydroxy(lower)alkyl,

(f) hydroxy,

(g) lower alkoxy,

(h) halo(lower)alkoxy,

(i) lower alkylamino,

(j) di-lower alkylamino,

(k) lower alkanoyl, and

(l) aryl;

n is 0 or 1;

Q is selected from the group consisting of a single bond or carbonyl;

T, U, V and W are each independently selected from the group consisting of

(1) nitrogen, and

(2) methine,
wherein the methine group is unsubstituted or optionally substituted with a substituent selected from the group consisting of

(a) halogen,

(b) lower alkyl,

(c) hydroxy, and

(d) lower alkoxy; and

wherein at least two of T, U, V, and W are methine;

X is selected from the group consisting of

(1) nitrogen, and

(2) methine; and

Y is selected from the group consisting of

(1) imino, unsubstituted or optionally substituted with lower alkyl, and

(2) oxygen.

In a subclass of this class, the anti-obesity agent is a NPY5 antagonist selected from the group consisting of:
(1) 3-oxo-N-(5-phenyl-2-pyrazinyl)-spiro[isobenzofuran-1(3H), 4′-piperidine]-1′-carboxamide,
(2) 3-oxo-N-(7-trifluoromethylpyrido[3,2-b]pyridin-2-yl)spiro-[isobenzofuran-1(3H), 4′-piperidine]-1′-carboxamide,
(3) N-[5-(3-fluorophenyl)-2-pyrimidinyl]-3-oxospiro-[isobenzofuran-1(3H), 4′-piperidine]-1′-carboxamide,
(4) trans-3′-oxo-N-(5-phenyl-2-pyrimidinyl)spiro[cyclohexane-1,1′ (3′H)-isobenzofuran]-4-carboxamide,
(5) trans-3′-oxo-N-[1-(3-quinolyl)-4-imidazolyl]spiro[cyclohexane-1,1′ (3′H)-isobenzofuran]-4-carboxamide,
(6) trans-3-oxo-N-(5-phenyl-2-pyrazinyl)spiro[4-azaiso-benzofuran-1(3H), 1′-cyclohexane]-4′-carboxamide,
(7) trans-N-[5-(3-fluorophenyl)-2-pyrimidinyl]-3-oxospiro[5-azaisobenzofuran-1(3H),1′-cyclohexane]-4′-carboxamide,
(8) trans-N-[5-(2-fluorophenyl)-2-pyrimidinyl]-3-oxospiro[5-azaisobenzofuran-1(3H), 1′-cyclohexane]-4′-carboxamide,
(9) trans-N-[1-(3,5-difluorophenyl)-4-imidazolyl]-3-oxospiro[7-azaisobenzofuran-1(3H), 1′-cyclohexane]-4′-carboxamide,
(10) trans-3-oxo-N-(1-phenyl-4-pyrazolyl)spiro[4-azaisobenzofuran-1(3H), 1′-cyclohexane]-4′-carboxamide,
(11) trans-N-[1-(2-fluorophenyl)-3-pyrazolyl]-3-oxospiro[6-azaisobenzofuran-1(3H), 1′-cyclohexane]-4′-carboxamide,
(12) trans-3-oxo-N-(1-phenyl-3-pyrazolyl)spiro[6-azaisobenzofuran-1 (3H), 1′-cyclohexane]-4′-carboxamide, and
(13) trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof.

In another subclass of this class, the NPY5 antagonist is Compound B, which corresponds to 3-oxo-N-(5-phenyl-2-pyrazinyl)-spiro[isobenzofuran-1(3H), 4′-piperidine]-1′-carboxamide, and pharmaceutically acceptable salts and esters thereof. In a subclass of this subclass, the composition comprises PYY3-36, or a salt or ester thereof; and 3-oxo-N-(5-phenyl-2-pyrazinyl)-spiro[isobenzofuran-1(3H), 4′-piperidine]-1′-carboxamide, and pharmaceutically acceptable salts and esters thereof.

In yet another subclass of this class, the NPY5 antagonist is 3-oxo-N-(7-trifluoromethylpyrido[3,2-b]pyridin-2-yl)spiro-[isobenzofuran-1(3H), 4′-piperidine]-1′-carboxamide, and pharmaceutically acceptable salts and esters thereof. In yet another subclass of this class, the NPY5 antagonist is N-[5-(3-fluorophenyl)-2-pyrimidinyl]-3-oxospiro-[isobenzofuran-1(3H), 4′-piperidine]-1′-carboxamide, and pharmaceutically acceptable salts and esters thereof. In yet another subclass of this class, the NPY5 antagonist is trans-3′-oxo-N-(5-phenyl-2-pyrimidinyl)spiro[cyclohexane-1,1′ (3′H)-isobenzofuran]-4-carboxamide, and pharmaceutically acceptable salts and esters thereof. In yet another subclass of this class, the NPY5 antagonist is trans-3′-oxo-N-[1-(3-quinolyl)-4-imidazolyl]spiro-[cyclohexane-1,1′(3′H)-isobenzofuran]-4-carboxamide, and pharmaceutically acceptable salts and esters thereof. In yet another subclass of this class, the NPY5 antagonist is trans-3-oxo-N-(5-phenyl-2-pyrazinyl)spiro[4-azaiso-benzofuran-1(3H),1′-cyclohexane]-4′-carboxamide, and pharmaceutically acceptable salts and esters thereof. In yet another subclass of this class, the NPY5 antagonist is trans-N-[5-(3-fluorophenyl)-2-pyrimidinyl]-3-oxospiro[5-azaisobenzofuran-1(3H),1′-cyclohexane]-4′-carboxamide, and pharmaceutically acceptable salts and esters thereof. In yet another subclass of this class, the NPY5 antagonist is trans-N-[5-(2-fluorophenyl)-2-pyrimidinyl]-3-oxospiro[5-azaisobenzofuran-1(3H),1′-cyclohexane]-4′-carboxamide, and pharmaceutically acceptable salts and esters thereof. In yet another subclass of this class, the NPY5 antagonist is trans-N-[1-(3,5-difluorophenyl)-4-imidazolyl]-3-oxospiro[7-azaisobenzofuran-1(3H), 1′-cyclohexane]-4′-carboxamide, and pharmaceutically acceptable salts and esters thereof. In yet another subclass of this class, the NPY5 antagonist is trans-3-oxo-N-(1-phenyl-4-pyrazolyl)spiro[4-azaisobenzofuran-1(3H),1′-cyclohexane]-4′-carboxamide, and pharmaceutically acceptable salts and esters thereof. In yet another subclass of this class, the NPY5 antagonist is trans-N-[1-(2-fluorophenyl)-3-pyrazolyl]-3-oxospiro[6-azaisobenzofuran-1(3H), 1′-cyclohexane]-4′-carboxamide, and pharmaceutically acceptable salts and esters thereof. In yet another subclass of this class, the NPY5 antagonist is trans-3-oxo-N-(1-phenyl-3-pyrazolyl)spiro[6-azaisobenzofuran-1(3H),1′-cyclohexane]-4′-carboxamide, and pharmaceutically acceptable salts and esters thereof. In yet another subclass of this class, the NPY5 antagonist is trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof. In yet another subclass of this class, the NPY5 antagonist is 3-oxo-N-(5-phenyl-2-pyrazinyl)-spiro[isobenzofuran-1(3H), 4′-piperidine]-1′-carboxamide, and pharmaceutically acceptable salts and esters thereof.

The NPY5 antagonists of formula I, including Compound B, and their preparation are disclosed in U.S. Pat. Nos. 6,326,375; 6,335,345; and International Publication No. WO 01/14376.

In another embodiment of the present invention, the anti-obesity agent is a NPY4 agonist, or a pharmaceutically acceptable salt or ester thereof. In a class of this embodiment, the NPY4 agonist is selected from the group consisting of: pancreatic peptide, and pharmaceutically acceptable salts or esters thereof. In another class of this embodiment, the composition comprises PYY3-36, or a salt or ester thereof; and pancreatic peptide, or a salt or ester thereof.

The present invention further relates to methods of treating or preventing obesity in a subject in need thereof by administering an effective amount of PYY, PYY₃₋₃₆ or a PYY agonist, and a second anti-obesity agent. Administration may be in a fixed combination or via co-administration of separate compositions or formulations. The present invention also relates to pharmaceutical compositions, and medicaments useful for carrying out these methods.

In another embodiment of the present invention, the invention comprises a method of treating a subject having a disorder associated with excessive food intake comprising administration of (a) PYY, PYY₃₋₃₆ or a PYY agonist, or a pharmaceutically acceptable salt or ester thereof; and (b) a therapeutically effective amount of an anti-obesity agent selected from the group consisting of: 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; aminorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; zonisamide; and topiramate; and pharmaceutically acceptable salts and esters thereof; to a subject in need of such treatment.

In a class of this embodiment, the method comprises administration of a therapeutically effective amount of PYY₃₋₃₆, or a pharmaceutically acceptable salt or ester thereof; and an anti-obesity agent, or a pharmaceutically acceptable salt or ester thereof.

In a subclass of this class, the anti-obesity agent is selected from the group consisting of: a NPY5 antagonist, and pharmaceutically acceptable salts or esters thereof.

In a subclass of this class, the NPY5 antagonist is selected from the group consisting of:
(1) 3-oxo-N-(5-phenyl-2-pyrazinyl)-spiro[isobenzofuran-1(3H), 4′-piperidine]-1′-carboxamide;
(2) 3-oxo-N-(7-trifluoromethylpyrido[3,2-b]pyridin-2-yl)spiro-[isobenzofuran-1(3H), 4′-piperidine]-1′-carboxamide;
(3) N-[5-(3-fluorophenyl)-2-pyrimidinyl]-3-oxospiro-[isobenzofuran-1(3H), 4′-piperidine]-1′-carboxamide;
(4) trans-3′-oxo-N-(5-phenyl-2-pyrimidinyl) spiro[cyclohexane-1,1′ (3′H)-isobenzofuran]-4-carboxamide;
(5) trans-3′-oxo-N-[1-(3-quinolyl)-4-imidazolyl]spiro[cyclohexane-1,1′ (3′H)-isobenzofuran]-4-carboxamide;
(6) trans-3-oxo-N-(5-phenyl-2-pyrazinyl)spiro[4-azaiso-benzofuran-1(3H), 1′-cyclohexane]-4′-carboxamide;
(7) trans-N-[5-(3-fluorophenyl)-2-pyrimidinyl]-3-oxospiro[5-azaisobenzofuran-1(3H), 1′-cyclohexane]-4′-carboxamide;
(8) trans-N-[5-(2-fluorophenyl)-2-pyrimidinyl]-3-oxospiro[5-azaisobenzofuran-1(3H), 1′-cyclohexane]-4′-carboxamide;
(9) trans-N-[1-(3,5-difluorophenyl)-4-imidazolyl]-3-oxospiro[7-azaisobenzofuran-1(3H), 1′-cyclohexane]-4′-carboxamide;
(10) trans-3-oxo-N-(1-phenyl-4-pyrazolyl)spiro[4-azaisobenzofuran-1(3H), 1′-cyclohexane]-4′-carboxamide;
(11) trans-N-[1-(2-fluorophenyl)-3-pyrazolyl]-3-oxospiro[6-azaisobenzofuran-1(3H), 1′-cyclohexane]-4′-carboxamide;
(12) trans-3-oxo-N-(1-phenyl-3-pyrazolyl)spiro[6-azaisobenzofuran-1(3H), 1′-cyclohexane]-4′-carboxamide; and
(13) trans-3-oxo-N-(2-phenyl-1,2,3-triazol-4-yl)spiro[6-azaisobenzofuran-1(3H),1′-cyclohexane]-4′-carboxamide;
   and pharmaceutically acceptable salts and esters thereof.

In another class of this embodiment, the disorder associated with excessive food intake is obesity.

In another class of this embodiment, the disorder associated with excessive food intake is an obesity-related disorder selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; metabolic syndrome; and acute lymphoblastic leukemia.

In a subclass of this class, the obesity-related disorder is diabetes.

In another subclass of this class, the obesity-related disorder is metabolic syndrome.

In another embodiment of the present invention, the invention comprises a method of treating a subject having a disorder associated with excessive food intake comprising administration of

(a) PYY, PYY₃₋₃₆ or a PYY agonist, or a pharmaceutically acceptable salt or ester thereof; and

(b) a therapeutically effective amount of an anti-obesity agent, wherein the anti-obesity agent is a MC4R agonist selected from the group consisting of:

(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide;
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
   and pharmaceutically acceptable salts and esters thereof.

In another class of this embodiment, the disorder associated with excessive food intake is obesity.

In another class of this embodiment, the disorder associated with excessive food intake is an obesity-related disorder selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; metabolic syndrome; and acute lymphoblastic leukemia.

In a subclass of this class, the obesity-related disorder is diabetes.

In another subclass of this class, the obesity-related disorder is metabolic syndrome.

In another embodiment of the present invention, the invention comprises a method of treating a subject having a disorder associated with excessive food intake comprising administration of

(a) PYY, PYY₃₋₃₆ or a PYY agonist, or a pharmaceutically acceptable salt or ester thereof; and

(b) a therapeutically effective amount of an anti-obesity agent, wherein the anti-obesity agent is a CB-1 antagonist/inverse agonist selected from the group consisting of:

(1) AM 251; and

(2) rimonabant;

and pharmaceutically acceptable salts and esters thereof.

In another class of this embodiment, the disorder associated with excessive food intake is obesity.

In another class of this embodiment, the disorder associated with excessive food intake is an obesity-related disorder selected from: overeating; bulimia; hypertension; diabetes, elevated plasma insulin concentrations; insulin resistance; dyslipidemia; hyperlipidemia; endometrial, breast, prostate and colon cancer; osteoarthritis; obstructive sleep apnea; cholelithiasis; gallstones; coronary heart disease; abnormal heart rhythms; heart arrythmias; myocardial infarction; polycystic ovarian disease; craniopharyngioma; the Prader-Willi Syndrome; Frohlich's syndrome; GH-deficient subjects; normal variant short stature; Turner's syndrome; metabolic syndrome; and acute lymphoblastic leukemia.

In a subclass of this class, the obesity-related disorder is diabetes.

In another subclass of this class, the obesity-related disorder is metabolic syndrome.

In another embodiment of the present invention, the invention comprises a method of maintaining weight loss in a subject comprising administration of

(a) a therapeutically effective amount of PYY, PYY₃₋₃₆, or a PYY agonist, and pharmaceutically acceptable salts and esters thereof; and

(b) a therapeutically effective amount of an anti-obesity agent selected from the group consisting of: 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; a minorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; zonisamide; and topiramate; and pharmaceutically acceptable salts and esters thereof; to a subject in need of such treatment.

In another embodiment of the present invention, the invention comprises a method of maintaining weight loss in a subject in need thereof comprising administration to said subject

(a) a therapeutically effective amount of PYY, PYY₃₋₃₆, or a PYY agonist, or a pharmaceutically effective salt or ester thereof; and

(b) a therapeutically effective amount of an anti-obesity agent, wherein the obesity agent is a MC4R agonist selected from the group consisting of:

(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide;
(7) 4-[2-(2-azetidin-1-yl-1(S)-methyl-2-oxoethyl)-4-chlorophenyl]-1-{[(3S,4R)-1-ter-t-butyl-4-(2,4-difluorophenyl)pyrrolidin-3-yl]carbonyl}piperidine;
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
   and pharmaceutically acceptable salts thereof.

In another embodiment of the present invention, the invention comprises a method of maintaining weight loss in a subject in need thereof comprising administration to said subject

(a) a therapeutically effective amount of PYY, PYY₃₋₃₆, or a PYY agonist, or a pharmaceutically effective salt or ester thereof; and

(b) a therapeutically effective amount of an anti-obesity agent, wherein the obesity agent is a CB-1 antagonist/inverse agonist selected from the group consisting of:

(1) AM 251; and

(2) Rimonabant;

and pharmaceutically acceptable salts or esters thereof.

In another embodiment of the present invention, the invention comprises a method of preventing obesity in a subject at risk for obesity comprising administration to said subject

(a) a prophylactically effective amount of PYY, PYY₃₋₃₆, or a PYY agonist, or a pharmaceutically effective salt or ester thereof; and

(b) a prophylactically effective amount of an anti-obesity agent selected from the group consisting of: 5HT transporter inhibitor; NE transporter inhibitor; ghrelin antagonist; H3 antagonist/inverse agonist; MCHlR antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; aminorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; zonisamide; and topiramate; or a pharmaceutically acceptable salt or and ester thereof.

In a class of this embodiment, the method of preventing obesity in a subject at risk for obesity comprises administration of a composition comprising PYY₃₋₃₆, or a pharmaceutically acceptable salt or ester thereof.

In another class of this embodiment, the method of preventing obesity in a subject at risk for obesity comprises administration of a composition comprising an anti-obesity, wherein the anti-obesity agent is a NPY5 antagonist selected from the group consisting of:
(1) 3-oxo-N-(5-phenyl-2-pyrazinyl)-spiro[isobenzofuran-1(3H), 4′-piperidine]-1′-carboxamide;
(2) 3-oxo-N-(7-trifluoromethylpyrido[3,2-b]pyridin-2-yl)spiro-[isobenzofuran-1(3H), 4′-piperidine]-1′-carboxamide;
(3) N-[5-(3-fluorophenyl)-2-pyrimidinyl]-3-oxospiro-[isobenzofuran-1(3H), 4′-piperidine]-1′-carboxamide;
(4) trans-3′-oxo-N-(5-phenyl-2-pyrimidinyl)spiro[cyclohexane-1,1′ (3′ H)-isobenzofuran]-4-carboxamide;
(5) trans-3′-oxo-N-[1-(3-quinolyl)-4-imidazolyl]spiro[cyclohexane-1,1′ (3′H)-isobenzofuran]-4-carboxamide;
(7) trans-3-oxo-N-(5-phenyl-2-pyrazinyl)spiro[4-azaiso-benzofuran-1(3H), 1′-cyclohexane]-4′-carboxamide;
(8) trans-N-[5-(3-fluorophenyl)-2-pyrimidinyl]-3-oxospiro[5-azaisobenzofuran-1(3H), 1′-cyclohexane]-4′-carboxamide;
(9) trans-N-[5-(2-fluorophenyl)-2-pyrimidinyl]-3-oxospiro[5-azaisobenzofuran-1(3H), 1′-cyclohexane]-4′-carboxamide;
(10) trans-N-[1-(3,5-difluorophenyl)-4-imidazolyl]-3-oxospiro[7-azaisobenzofuran-1(3H), 1′-cyclohexane]-4′-carboxamide;
(11) trans-3-oxo-N-(1-phenyl-4-pyrazolyl)spiro[4-azaisobenzofuran-1(3H),1′-cyclohexane]-4′-carboxamide;
(12) trans-N-[1-(2-fluorophenyl)-3-pyrazolyl]-3-oxospiro[6-azaisobenzofuran-1(3H), 1′-cyclohexane]-4′-carboxamide;
(13) trans-3-oxo-N-(1-phenyl-3-pyrazolyl)spiro[6-azaisobenzofuran-1(3H), 1′-cyclohexane]-4′-carboxamide; and
(14) trans-3-oxo-N-(2-phenyl-1,2,3-triazol-4-yl)spiro[6-azaisobenzofuran-1(3H),1′-cyclohexane]-4′-carboxamide;
   and pharmaceutically acceptable salts and esters thereof.

In another embodiment of the present invention, the invention comprises a method of preventing obesity in a subject at risk for obesity comprising administration to said subject

(a) a prophylactically effective amount of PYY, PYY₃₋₃₆, or a PYY agonist, or a pharmaceutically effective salt or ester thereof; and

(b) a prophylactically effective amount of an anti-obesity agent, wherein the obesity agent is a MC4R agonist selected from the group consisting of:

(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide;
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
   and pharmaceutically acceptable salts thereof.

In another embodiment of the present invention, the invention comprises a method of preventing obesity in a subject at risk for obesity comprising administration to said subject

(a) a prophylactically effective amount of PYY, PYY₃₋₃₆, or a PYY agonist, or a pharmaceutically effective salt or ester thereof; and

(b) a prophylactically effective amount of an anti-obesity agent, wherein the obesity agent is a CB-1 antagonist/inverse agonist selected from the group consisting of:

(1) AM 251; and

(2) Rimonabant;

and pharmaceutically acceptable salts or esters thereof.

In another embodiment of the present invention, the invention comprises the use of PYY, PYY₃₋₃₆, or a PYY agonist, or a pharmaceutically acceptable salt or ester thereof; and an anti-obesity agent selected from the group consisting of: 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; a minorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; zonisamide; and topiramate; or a pharmaceutically acceptable salt or ester thereof;

for the manufacture of a medicament for treatment of obesity which comprises an effective amount of PYY, PYY₃₋₃₆, or a PYY agonist, and an effective amount of anti-obesity agent, together or separately.

In another embodiment of the present invention, the invention comprises the use of PYY, PYY₃₋₃₆, or a PYY agonist, or a pharmaceutically acceptable salt or ester thereof; and an anti-obesity agent, wherein the anti-obesity agent is a MC4R agonist selected from the group consisting of:
(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide;
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) Methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
   and pharmaceutically acceptable salts and esters thereof;
   for the manufacture of a medicament for treatment of obesity which comprises an effective amount of PYY, PYY₃₋₃₆, or a PYY agonist, and an effective amount of the MC4R agonist, together or separately.

In another embodiment of the present invention, the invention comprises the use of PYY, PYY₃₋₃₆, or a PYY agonist, or a pharmaceutically acceptable salt or ester thereof; and an anti-obesity agent, wherein the anti-obesity agent is a CB-1 antagonist/inverse agonist selected from the group consisting of:

(1) AM 251; and

(2) Rimonabant;

and pharmaceutically acceptable salts and esters thereof;

for the manufacture of a medicament for treatment of obesity which comprises an effective amount of PYY, PYY₃₋₃₆, or a PYY agonist, and an effective amount of the CB-1 antagonist/inverse agonist, together or separately.

The present invention further relates to the use of PYY, PYY₃₋₃₆ or a PYY agonist, and an anti-obesity agent for the manufacture of a medicament for treatment of obesity which comprises an effective amount of PYY, PYY₃₋₃₆ or the PYY agonist, and an effective amount of anti-obesity agent, together or separately.

The present invention further relates to a product containing PYY, PYY₃₋₃₆ or a PYY agonist, and an anti-obesity agent as a combined preparation for simultaneous, separate or sequential use in obesity.

As used herein, the term “anti-obesity agent” includes compounds that reduce total food intake by 5 to 30%, or reduce caloric intake or selectively reduce intake of specific components of the diet such as carbohydrates or fats by 5 to 30%; compounds which, when administered to a subject, act to increase the metabolic rate of the subject, particularly those agents which increase metabolic rate by at least 5%, preferably 10%, most preferably 20% in 24 hour energy expenditure when administered to the subject; and compounds that inhibit the absorption of 10 to 50% of the nutrients.

One of ordinary skill in the art can readily identify anti-obesity agents useful in the compositions and methods of the present invention. Anti-obesity agents that decrease food intake can be evaluated in rodents according to the procedures described in: Daniels, A. J. et al., Regulatory Peptides, 106: 47-54 (2002); Halaas, J. L. et. al., Science, 269: 543-546 (1995); and Strack, A. M., Obesity Research, 10: 173-81 (2002). Anti-obesity agents that increase metabolic rate are routinely evaluated in rodents (Atgie, C., Comp. Biochem. Physiol. A. Mol. Integr. Physiol. 119: 629-36 (1998); Hinims-Hagan, J., American J. Physiology, 266:R1371-82 (1994)), and, even when inactive in rodents, are tested in additional species such as dog and monkey before ultimately being tested in humans (Connacher, A. A. et. al., Int'l J. Obesity, 16: 685-694 (1992); Connacher, A. A. et. al., Am. J. Clin. Nutr., 55: 258S-261S (1992); Connacher, A. A. et. al., Brit. Med. J., 296: 1217-1220 (1998)). The utility of anti-obesity agents that enhance metabolic rate is supported by experiments with mice, in which the R11-beta gene has been deleted, that were shown to be resistant to diet induced obesity (D. E. Cummings et al. Nature 382: 622-626 (1996)). Anti-obesity agents that inhibit nutrient absorption can be evaluated in: Badr M. Z. and Chen, T. S., Toxicology, 34: 333-40 (1985); Sorribas, V., J. Pharm. Pharmacol., 44: 1030-2 (1992).

Serotonin (5HT) transport inhibitors useful in this invention include, but are not limited to, paroxetine, fluoxetine, fenfluramine, fluvoxamine, sertraline, and imipramine.

Norepinephrine (NE) transport inhibitors useful in this invention include, but are not limited to, GW 320659, despiramine, talsupram, and nomifensine.

Cannabinoid receptor 1 (CB-1) antagonists/inverse agonists useful in the present invention include: U.S. Pat. Nos. 5,532,237, 4,973,587, 5,013,837, 5,081,122, 5,112,820, 5,292,736, 5,624,941 and U.S. Pat. No. 6,028,084; US Patent Publication No. US 2004/048317, and US 2004/0058820, which are incorporated by reference herein; and PCT Application Nos. WO 96/33159, WO 98/33765, WO98/43636, WO98/43635, WO 01/09120, WO 01.96330, WO98/31227, WO98/41519, WO98/37061, WO00/10967, WO00/10968, WO97/29079, WO99/02499, WO 01/58869, WO 02/076949, WO 01/64632, WO 01/64633, WO 01/64634, WO 03/006007, WO 03/077847; WO 03/007887, and WO 2004/049317; and EPO Application No. EP-658546. Specific CB-1 antagonists/inverse agonists useful in the present invention include, but are not limited to, rimonabant (Sanofi Synthelabo), SR-147778 (Sanofi Synthelabo), BAY 65-2520 (Bayer), and SLV 319 (Solvay).

Ghrelin antagonists useful in the present invention, include, but are not limited to, those disclosed in: PCT Application Nos. WO 01/87335, and WO 02/08250.

Histamine 3 (H3) antagonist/inverse agonists useful in the present invention include, but are not limited to, those disclosed in: PCT Application No. WO 02/15905; and O-[3-(1H-imidazol-4-yl)propanol]carbamates (Kiec-Kononowicz, K. et al., Pharmazie, 55: 349-55 (2000)), piperidine-containing histamine H3-receptor antagonists (Lazewska, D. et al., Pharmazie, 56: 927-32 (2001), benzophe-none derivatives and related compounds (Sasse, A. et al., Arch. Pharm. (Weinheim) 334: 45-52 (2001)), substituted N-phenylcarbamates (Reidemeister, S. et al., Pharmazie, 55: 83-6 (2000)), and proxifan derivatives (Sasse, A. et al., J. Med. Chem. 43: 3335-43 (2000)). Specific H3 antagonists/inverse agonists useful in the present invention include, but are not limited to, thioperamide, 3-(1H-imidazol-4-yl)propyl N-(4-pentenyl)carbamate, clobenpropit, iodophenpropit, imoproxifan, and GT2394 (Gliatech).

Melanin-concentrating hormone 1 receptor (MCH1R) antagonists useful in the present invention include, but are not limited to, those disclosed in: Melanin-concentrating hormone 1 receptor (MCH1R) antagonists, such as T-226296 (Takeda), SNP-7941 (Synaptic), and the compounds disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027, and Japanese Patent Application Nos. JP 13226269, and JP 2004-139909.

Neuropeptide Y1 (NPY1) antagonists useful in the present invention, include, but are not limited to, those disclosed in: U.S. Pat. No. 6,001,836; and PCT Application Nos. WO 96/14307, WO 01/23387, WO 99/51600, WO 01/85690, WO 01/85098, WO 01/85173, and WO 01/89528. Specific examples of NPY1 antagonists useful in the present invention include, but are not limited to, BMBP3226, J-115814, BIBO 3304, LY-357897, CP-671906, and GI-264879A.

Other NPY5 antagonists useful in the present invention, include, but are not limited to, the compounds disclosed in: U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175. Specific NPY 5 antagonists useful in the combinations of the present invention, include, but are not limited to GW-569180A, GW-594884A, GW-587081×, GW-548118X; FR226928, FR 240662, FR252384; 1229U91, GI-264879A, CGP71683A, LY-377897, PD-160170, SR-120562A, SR-120819A and JCF-104. Additional specific NPY 5 antagonists useful in the combinations of the present invention, include, but are not limited to the compounds described in Norman et al., J. Med. Chem. 43: 4288-4312 (2000).

One of ordinary skill in the art, can readily identify other NPY5 antagonist compounds useful in the compositions and methods of the present invention using the methods described in WO 96/16542. NPY5 antagonists which are useful in the present invention generally have an IC₅₀ less than 1 uM in the NPY Y5 binding assay described in Kanatani et al., Biochem. Biophys. Res. Commun. 272: 169-173 (2000). NPY5 antagonists which are preferred in the present invention generally have an IC₅₀ less than 100 nM in the NPY Y5 binding assay.

Neuropeptide Y4 (NPY4) agonists useful in the present invention, include, but are not limited to, compounds such as pancreatic peptide (PP) as described in Batterham et al., J. Clin. Endocrinol. Metab. 88: 3989-3992 (2003), and other Y4 agonists such as 1229U91 (Raposinho et al., Neuroendocrinology. 71: 2-7 (2000)).

Leptin includes, but is not limited to, recombinant human leptin (PEG-OB, Hoffman La Roche) and recombinant methionyl human leptin (Amgen). Leptin derivatives (e.g., truncated forms of leptin) useful in the present invention include, but are not limited to, those disclosed in: U.S. Pat. Nos. 5,552,524; 5,552,523; 5,552,522; 5,521,283; and PCT International Publication Nos. WO 96/23513; WO 96/23514; WO 96/23515; WO 96/23516; WO 96/23517; WO 96/23518; WO 96/23519; and WO 96/23520.

Opioid antagonists useful in the present invention include, but are not limited to, those disclosed in: PCT Application No. WO 00/21509. Specific opioid antagonists useful in the present invention include, but are not limited to, nalmefene (Revex®), 3-methoxynaltrexone, naloxone, and naltrexone.

Orexin antagonists useful in the present invention include, but are not limited to, those disclosed in: PCT Patent Application Nos. WO 01/96302, WO 01/68609, WO 02/51232, and WO 02/51838. Specific orexin antagonists useful in the present invention include, but are not limited to, SB-334867-A.

An acyl-estrogen useful in the present invention include oleoyl-estrone (del Mar-Grasa, M. et al., Obesity Research, 9: 202-9 (2001)).

11β HSD-1 (11-beta hydroxy steroid dehydrogenase type 1) inhibitor useful in the present invention include, but are not limited to, BVT 3498, BVT 2733, and those disclosed in WO 01/90091, WO 01/90090, WO 01/90092, and U.S. Pat. No. 6,730,690 and US Publication No. US 2004-0133011, which are incorporated by reference herein in their entirety.

Cholecystokinin-A (CCK-A) agonists useful in the present invention include, but are not limited to, those disclosed in U.S. Pat. No. 5,739,106. Specific CCK-A agonists include, but are not limited to, AR-R 15849, GI 181771, JMV-180, A-71378, A-71623 and SR146131.

Specific ciliary neurotrophic factors (CNTF) useful in the present invention include, but are not limited to, GI-181771 (Glaxo-SmithKline); SR146131 (Sanofi Synthelabo); butabindide; PD170,292, PD 149164 (Pfizer). CNTF derivatives useful in the present invention include, but are not limited to, axokine (Regeneron); and those disclosed in PCT Application Nos. WO 94/09134, WO 98/22128, and WO 99/43813.

Specific Cox-2 inhibitors useful in the present invention include, but are not limited to, rofecoxib (VIOXX®, see U.S. Pat. No. 5,474,995, hereby incorporated by reference in its entirety), etoricoxib (ARCOXIA™ see U.S. Pat. No. 5,861,419, hereby incorporated by reference in its entirety), celecoxib (CELEBREX®, see U.S. Pat. No. 5,466,823, hereby incorporated by reference in its entirety), valdecoxib (see U.S. Pat. No. 6,633,272, hereby incorporated by reference in its entirety), parecoxib (see U.S. Pat. No. 5,932,598, hereby incorporated by reference in its entirety), lumiracoxib (PREXIGE®, Novartis), BMS347070 (Bristol Myers Squibb), tiracoxib or JTE522 (Japan Tobacco), ABT963 (Abbott), CS502 (Sankyo) and GW406381 (GlaxoSmithKline), and pharmaceutically acceptable salts thereof.

DP-IV Inhibitors useful in the present invention include, but are not limited to, isoleucine thiazolidide, valine pyrrolidide, NVP-DPP728, LAF237, P93/01, TSL 225, TAC-2λ/2B/2C, FE 999011, P9310/K364, VIP 0177, SDZ 274-444; and the compounds disclosed in U.S. Pat. No. 6,699,871, which is incorporated herein by reference; and International Patent Application Nos. WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181.

Growth hormone secretagogue (GHS) agonists useful in the present invention include, but are not limited to, those disclosed in: U.S. Pat. No. 6,358,951, and U.S. Patent Application Nos. 2002/049196 and 2002/022637; and PCT Application Nos. WO 01/56592, and WO 02/32888. Specific GHS agonists include, but are not limited to, NN703, hexarelin, MK-0677, SM-130686, CP-424,391, L-692,429 and L-163,255.

5HT2C agonists useful in the present invention include, but are not limited to, those disclosed in: U.S. Pat. No. 3,914,250; and PCT Application Nos. WO 02/36596, WO 02/48124, WO 02/10169, WO 01/66548, WO 02/44152; WO 02/51844, WO 02/40456, and WO 02/40457. Specific 5HT2C agonists useful in this invention include, but are not limited to, BVT933, DPCA37215, WAY161503, and R-1065.

Mc4r agonists useful in the present invention include, but are not limited to, those disclosed in: U.S. Pat. Nos. 6,410,548; 6,294,534; 6,350,760; 6,458,790; 6,472,398; and 6,376,509; and PCT Application Nos. WO 99/64002; WO 00/74679; WO 01/70708; WO 01/70337; WO 01/91752; WO 02/059095; WO 02/059107; WO 02/059108; WO 02/059117; WO 02/068387; WO 02/068388; WO 02/067869; WO 03/007949; WO 03/009847; WO2004/078717; WO2004/089307; and WO2005/009950. Specific Mc4r agonists useful in the present invention include CEIR86036 (Chiron); ME-10142; ME-10145 (Melacure); MT-II, PT-141, and PT-14 (Palatin).

Monoamine reuptake inhibitors useful in the present invention include, but are not limited to, those disclosed in: PCT Application Nos. WO 01/27068, and WO 01/62341. Specific monoamine reuptake inhibitors useful in the present invention include, but are not limited to, sibutramine (Meridia®/Reductil®) disclosed in U.S. Pat. Nos. 4,746,680, 4,806,570, and 5,436,272, and U.S. Patent Publication No. 2002/0006964 The present invention encompasses sibutramine as a racemic mixture, as optically pure isomers (+) and (−), or a pharmaceutically acceptable salt, solvent, hydrate, clathrate or prodrug thereof; particularly sibutramine hydrochloride monohydrate.

Serotonin reuptake inhibitors useful in the present invention include, but are not limited to, those disclosed in: U.S. Pat. No. 6,365,633; and PCT Patent Application Nos. WO 01/27060, and WO 01/162341.

Uncoupling Protein (UCP-1, UCP-2, and UCP-3) activators useful in the present invention include, but are not limited to, those disclosed in: PCT Patent Application No. WO 99/00123. Specific uncoupling protein (UCP-1, UCP-2, and UCP-3) activators useful in the present invention include, but are not limited to, phytanic acid, 4-[(E)-2-(5,6,7,8-tetrahydro-5,5,8,8-tetramethyl-2-napthalenyl)-1-propenyl]benzoic acid (TTNPB), and retinoic acid.

β3 adrenergic receptor (β3) agonists useful in the present invention include, but are not limited to, those disclosed in: U.S. Pat. Nos. 5,705,515, and 5,451,677; and PCT Patent Application Nos. WO 01/74782, and WO 02/32897. Specific β3 agonists useful in the present invention include, but are not limited to, AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A.

Thyroid hormone β agonists useful in the present invention include, but are not limited to, those disclosed in: PCT Application No. WO 02/15845; and Japanese Patent Application No. JP 2000256190. Specific thyroid hormone β agonists useful in the present invention include, but are not limited to, KB-2611 (KaroBioBMS).

Specific fatty acid synthase (FAS) inhibitors useful in the present invention, include, but are not limited to, Cerulenin and C75.

Specific phosphodieterase (PDE) inhibitors useful in the present invention, include, but are not limited to, theophylline, pentoxifylline, zaprinast, sildenafil, anrinone, milrinone, cilostamide, rolipram, and cilomilast.

Lipase inhibitors useful in the present invention include, but are not limited to, those disclosed in: PCT Application No. WO 01/77094. Specific lipase inhibitors useful in the present invention include, but are not limited to, orlistat (Xenical®), Triton WR1339, RHC80267, lipstatin, tetrahydrolipstatin, teasaponin, and diethylumbelliferyl phosphate.

Topiramate (Topimax®), indicated as an anti-convulsant and an anti-convulsant, has been shown to increase weight loss. Metformin (Glucophage®) is indicated for patients with non-insulin dependent diabetes mellitus, particularly those with refractory obesity. Physician's Desk Reference® page 1080-1086, (56^{th} ed. 2002).

Specific bombesin (BRS-3) agonists useful in the present invention, include, but are not limited to, [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75).

Zonisamide, a marketed antiepileptic drug with serotonergic and dopaminergic activity in addition to the ability to block sodium and calcium channels, has been shown to result in weight loss in epileptic adults and in obese adults.

The above compounds are only illustrative of the PYY agonists and anti-obesity agents that can be used in the compositions of the present invention. As this listing of compounds is not meant to be comprehensive, the methods of the present invention may employ any PYY agonist, and any anti-obesity agent, and are not limited to any particular structural class of compounds.

The present invention also relates to the treatment of obesity with a combination of PYY, PYY₃₋₃₆ or a PYY agonist, and an anti-obesity agent which may be administered separately, therefore the invention also relates to combining separate pharmaceutical compositions or formulations into a kit form. The kit, according to this invention, comprises two separate pharmaceutical compositions: a first unit dosage form comprising a prophylactically or therapeutically effective amount of PYY, PYY₃₋₃₆ or a PYY agonist, or a pharmaceutically acceptable salt or ester thereof, and a pharmaceutically acceptable carrier or diluent, and a second unit dosage form comprising a prophylactically or therapeutically effective amount of another anti-obesity agent, or a pharmaceutically acceptable salt or ester thereof, and a pharmaceutically acceptable carrier or diluent, and a container. The routes of administration of the unit dosage forms within the kit may differ. For example, PYY, PYY₃₋₃₆ and the PYY agonist in the first unit dosage form may be administered by inhalation using a nasal spray, or by injection via a syringe; whereas the anti-obesity agent in the second unit dosage form may be orally administered. The present invention also includes administration at different times and frequencies. The kit further comprises a container. Such kits are especially suited for the delivery of solid oral forms such as tablets or capsules, as well as nasal sprays and injectable formulations. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. Another example of a kit, it a multi container kit with a nasal spray bottle containing PYY, PYY3-36 or a PYY agonist, and oral dosage units (such as tablets or capsules) containing the second anti-obesity agent. If desired, a memory aid can be provided, for example in the form of numbers, letters, or other markings or with a calendar insert, designating the days or time in the treatment schedule in which the dosages can be administered.

“Halogen atom” refers to fluorine atom, chlorine atom, bromine atom and iodine atom.

“Lower alkyl” refers to a straight- or branched-chain alkyl group of C1 to C6, for example, methyl, ethyl, propyl, isopropyl, butyl, isobutyl, sec-butyl, tert-butyl, pentyl, isopentyl, hexyl, isohexyl, and the like.

“Halo(lower)alkyl” refers to the aforesaid lower alkyl substituted with 1 or more than 2, preferably 1 to 3 aforesaid halogen atoms identically or differently at the substitutable, arbitrary positions, for example, fluoromethyl, difluoromethyl, trifluoromethyl, 2-fluoroethyl, 1,2-difluoroetliyl, chloromethyl, 2-chloroethyl, 1,2-dichloroethyl, bromomethyl, iodomethyl, and the like.

“Hydroxy(lower)alkyl” refers to the aforesaid lower alkyl substituted with 1 or more than 2, preferably 1 or 2 hydroxy groups at the substitutable, arbitrary positions, for example, hydroxymethyl, 2-hydroxyethyl, 1-hydroxy-1-methylethyl, 1,2-dihydroxyethyl, 3-hydroxypropyl, and the like.

“Cyclo(lower)alkyl” refers to a cycloalkyl group of C3 to C6, for example, cyclopropyl, cyclobutyl, cyclopentyl, cyclohexyl, and the like.

“Lower alkenyl” refers to a straight- or branched-chain alkenyl group of C2 to C6, for example, vinyl, 1-propenyl, 2-propenyl, isopropenyl, 3-butenyl, 2-butenyl, 1-butenyl, 1-methyl-2-propenyl, 1-methyl-1-propenyl, 1-ethyl-1-ethenyl, 2-methyl-2-propenyl, 2-methyl-1-propenyl, 3-methyl-2-butenyl, 4-pentenyl, and the like.

“Lower alkoxy” refers to a straight- or branched-chain alkoxy group of C1 to C6, for example, methoxy, ethoxy, propoxy, isopropoxy, butoxy, sec-butoxy, isobutoxy, tert-butoxy, pentyloxy, isopentyloxy, hexyloxy, isohexyloxy, and the like.

“Halo(lower)alkoxy” refers to the aforesaid lower alkoxy substituted with 1 or more than 2, preferably 1 to 3 aforesaid halogen atoms identically or differently at the substitutable, arbitrary positions, for example, fluoromethoxy, difluoromethoxy, trifluoromethoxy, 2-fluoroethoxy, 1,2-difluoroethoxy, chloromethoxy, 2-chloroethoxy, 1,2-dichloroethoxy, bromomethoxy, iodomethoxy, and the like.

“Lower alkylthio” refers to a straight- or branched-chain alkylthio group of C1 to C6, for example, methylthio, ethylthio, propylthio, isopropylthio, butylthio, sec-butylthio, isobutylthio, tert-butylthio, pentylthio, isopentylthio, hexylthio, isohexylthio, and the like.

“Lower alkanoyl” refers to an alkanoyl group containing the aforesaid lower alkyl, that is, an alkanoyl group of C2 to C7, for example acetyl, propionyl, butyryl, isobutyryl, valeryl, isovaleryl, pivaloyl, and the like.

“Lower alkoxycarbonyl” refers to an alkoxycarbonyl group containing the aforesaid lower alkoxy, that is, an alkoxycarbonyl group of C2 to C7, for example, methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, and the like.

“Lower alkylene optionally substituted with oxo” refers to a straight- or branched-chain alkylene group of C2 to C6 which may be substituted with 1 or more than 2, preferably 1 oxo group at a substitutable, arbitrary position, for example, ethylene, trimethylene, tetramethylene, pentamethylene, hexamethylene, 1-oxoethylene, 1-oxotrimethylene, 2-oxotrimethylene, 1-oxotetramethylene, 2-oxotetramethylene, and the like.

“Aryl” includes phenyl, naphthyl, and the like.

“Heteroaryl” refers to 5- or 6-membered monocylic heteroaromatic group which contains 1 or more than 2, preferably 1 to 3 hetero atoms identically or differently selected from the group of oxygen atom, nitrogen atom and sulfur atom; or condensed heteroaromatic group, where the aforesaid monocylic heteroaromatic group is condensed with the aforesaid aryl group, or with the identified or different aforesaid monocylic heteroaromatic group each other, for example, pyrrolyl, furyl, thienyl, imidazolyl, pyrazolyl, thiazolyl, isothiazolyl, oxazolyl, isoxazolyl, 1,2,3-triazolyl, 1,2,4-triazolyl, tetrazolyl, oxadiazolyl, 1,2,3-thiadiazolyl, 1,2,4-thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzoxazolyl, benzisoxazolyl, benzothiazolyl, benzisothiazolyl, indazolyl, purinyl, quinolyl, isoquinolyl, phthalazyl, naphthylidinyl, quinoxalinyl, quinazolinyl, cinnolinyl, pteridinyl, pyrido[3,2-b]pyridyl, and the like.

“Lower alkylamino” refers to an amino group mono-substituted with the aforesaid lower alkyl, for example, methylamino, ethylamino, propylamino, isopropylamino, butylamino, sec-butylamino, tert-butylamino, and the like.

“Di-lower alkylamino” refers to an amino group di-substituted with identical or different aforesaid lower alkyl, for example, dimethylamino, diethylamino, ethylmethylamino, dipropylamino, methylpropylamino, diisopropylamino, and the like.

In order to disclose the aforesaid compounds of the general formula (I) more detailed, the various symbols used in the formula (I) are explained in more detail by the use of preferred embodiments.

Ar¹ represents aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar².

“Aryl or heteroaryl which may be substituted, the substituent being selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group represented by formula of -Q-Ar²” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or more than 2, preferably 1 or 2 selected from the group consisting of halogen, nitro, lower alkyl, halo(lower)alkyl, hydroxy(lower)alkyl, cyclo(lower)alkyl, lower alkenyl, lower alkoxy, halo(lower)alkoxy, lower alkylthio, carboxyl, lower alkanoyl, lower alkoxycarbonyl, lower alkylene optionally substituted with oxo, and a group of formula: -Q-Ar².

Halogen atom as the aforesaid substituent includes fluorine atom, chlorine atom, and the like preferably.

Lower alkyl as the aforesaid substituent includes methyl, ethyl, propyl, isopropyl, and the like preferably.

Halo(lower)alkyl as the aforesaid substituent includes difluoromethyl, trifluoromethyl, and the like preferably.

Hydroxy(lower)alkyl as the aforesaid substituent includes hydroxymethyl, 2-hydroxyethyl, 1-hydroxy-1-methylethyl, and the like preferably.

Cyclo(lower)alkyl as the aforesaid substituent includes cyclopropyl, cyclobutyl, and the like preferably.

Lower alkenyl as the aforesaid substituent includes vinyl, 1-propenyl, 2-methyl-1-propenyl, and the like preferably.

Lower alkoxy as the aforesaid substituent includes methoxy, ethoxy, and the like preferably.

Halo(lower)alkoxy as the aforesaid substituents includes fluoromethoxy, difluoromethoxy, trifluoromethoxy, and the like preferably.

Lower alkylthio as the aforesaid substituent includes methylthio, ethylthio, and the like preferably.

Lower alkanoyl as the aforesaid substituent includes acetyl, propionyl, and the like preferably.

Lower alkoxycarbonyl as the aforesaid substituent includes methoxycarbonyl, ethoxycarbonyl, and the like preferably.

Lower alkylene optionally substituted with oxo as the aforesaid substituent includes 1-oxotetramethylene, and the like preferably.

In a group of formula: -Q-Ar² as the aforesaid substituent, Ar² represents aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl;

Q represents a single bond or carbonyl.

“Aryl or heteroaryl which may be substituted, the substituent being selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl” refers to unsubstituted aforesaid aryl or aforesaid heteroaryl, or the aforesaid aryl or aforesaid heteroaryl which has substituent(s) at the substitutable, arbitrary position(s). The aforesaid substituent can be, identically or differently, one or not less than 2, preferably 1 or 2 selected from the group consisting of halogen, cyano, lower alkyl, halo(lower)alkyl, hydroxy(lower)alkyl, hydroxy, lower alkoxy, halo(lower)alkoxy, lower alkylamino, di-lower alkylamino, lower alkanoyl and aryl.

Halogen atom as the aforesaid substituent includes, preferably, fluorine atom, chlorine atom, and the like.

Lower alkyl as the aforesaid substituent includes, preferably, methyl, ethyl, propyl, isopropyl, and the like.

Halo(lower)alkyl as the aforesaid substituent includes, preferably, difluoromethyl, trifluoromethyl, and the like.

Hydroxy(lower)alkyl as the aforesaid substituent includes, preferably, hydroxymethyl, 2-hydroxyethyl, 1-hydroxy-1-methylethyl, and the like.

Lower alkoxy as the aforesaid substituent includes, preferably, methoxy, ethoxy, and the like.

Halo(lower)alkoxy as the aforesaid substituent includes, preferably, fluoromethoxy, difluoromethoxy, trifluoromethoxy, and the like.

Lower alkylamino as the aforesaid substituent includes, preferably, methylamino, ethylamino, and the like.

Di-lower alkylamino as the aforesaid substituent includes, preferably, dimethylamino, diethylamino, and the like.

Lower alkanoyl as the aforesaid substituent includes, preferably, acetyl, propionyl, and the like.

Aryl as the aforesaid substituent includes, preferably, phenyl, and the like.

The substituent(s) of Ar² include, preferably, halogen, cyano, lower alkyl, halo(lower)alkyl, hydroxy(lower)alkyl, hydroxy, halo(lower)alkoxy, and the like.

Aryl in Ar² includes, preferably, phenyl, and the like and heteroaryl includes imidazolyl, pyridyl, benzofuranyl, quinolyl, and the like. Consequently, a group of formula: -Q-Ar² includes, for example, phenyl, 2-fluorophenyl, 3-fluorophenyl, 4-fluorophenyl, 2,3-difluorophenyl, 2,4-difluorophenyl, 3,5-difluorophenyl, 2-chlorophenyl, 3-chlorophenyl, 4-chlorophenyl, 2-cyanophenyl, 3-cyanophenyl, 4-cyanophenyl, 2-methylphenyl, 3-methylphenyl, 4-methylphenyl, 2-fluoro-5-methylphenyl, 3-fluoromethylphenyl, 2-trifluoromethylphenyl, 3-trifluoromethylphenyl, 4-trifluoromethylphenyl, 2-methoxyphenyl, 3-methoxyphenyl, 4-methoxyphenyl, 3-fluoro-5-methoxyphenyl, 3-fluoromethoxyphenyl, 3-difluoromethoxyphenyl, 3-(2-hydroxyethyl)phenyl, 3-hydroxymethylphenyl, 3-(1-hydroxy-1-methylethyl)phenyl, 3-hydroxyphenyl, 4-hydroxyphenyl, 2-imidazolyl, 1-ethyl-2-imidazolyl, 1,2,4-thiadiazol-5-yl, 1,3,4-thiadiaol-2-yl, 2-pyridyl, 3-pyridyl, 4-pyridyl, 2-ethyl-4-pyridyl, 4-pyrimidinyl, 5-pyrimidinyl, 4-benzo[b]furanyl, 5-benzo[b]furanyl, 7-benzo[b]furanyl, 2-quinolyl, 3-quinolyl, 4-quinolyl, 5-quinolyl, 6-quinolyl, 8-quinolyl, benzoyl, 2-pyridylcarbonyl, and the like, and preferably, phenyl, 2-fluorophenyl, 3-fluorophenyl, 3,5-difluorophenyl, 3-chlorophenyl, 4-chlorophenyl, 3-cyanophenyl, 3-trifluoromethylphenyl, 3-difluoromethoxyphenyl, 3-(2-hydroxyethyl)phenyl, 3-hydroxyphenyl, 4-hydroxyphenyl, 1-ethyl-2-imidazolyl, 2-pyridyl, 7-benzo[b]furanyl, 2-quinolyl, 3-quinolyl, benzoyl, 2-pyridylcarbonyl, and the like.

The substituent of Ar¹ includes, preferably, halogen, lower alkyl, halo(lower)alkyl, lower alkenyl, lower alkanoyl, lower alkylene optionally substituted with oxo, and a group of formula: -Q-Ar², and the like.

Aryl in Ar¹ includes, preferably, phenyl, and the like and heteroaryl of Ar¹ includes pyrrolyl, imidazolyl, pyrazolyl, thiazolyl, oxazolyl, isoxazolyl, 1,2,3-triazolyl, 1,2,4-thiadiazolyl, pyridyl, pyrazinyl, pyrimidinyl, 1,2,4-triazinyl, benzoxazolyl, benzothiazolyl, quinolyl, pyrido[3,2-b]pyridyl, and the like. Consequently, Ar¹ includes, for example, 3-fluorophenyl, 4-fluorophenyl, 3,4-difluorophenyl, 3-chlorophenyl, 4-chlorophenyl, 3,4-dichlorophenyl, 4-acetylphenyl, 5-oxo-5,6,7,8-tetrahydro-2-naphthyl, 4-acetyl-3-trifluoromethylphenyl, 4-(1-ethyl-2-imidazolyl)phenyl, 3-(2-pyridyl)phenyl, 3-(4-pyridyl)phenyl, 4-(2-pyridyl)phenyl, 4-(3-pyridyl)phenyl, 4-(2-ethyl-4-pyridyl)phenyl, 4-(4-pyrimidinyl)phenyl, 4-benzoylphenyl, 4-(2-pyridylcarbonyl)phenyl, 1-phenyl-3-pyrrolyl, 1-phenyl-4-imidazolyl, 1-(2-fluorophenyl)-4-imidazolyl, 1-(3-fluorophenyl)-4-imidazolyl, 1-(4-fluorophenyl)-4-imidazolyl, 1-(2,3-difluorophenyl)-4-imidazolyl, 1-(2,4-difluorophenyl)-4-imidazolyl, 1-(3,5-difluorophenyl)-4-imidazolyl, 1-(3-chlorophenyl)-4-imidazolyl, 1-(2-cyanophenyl)-4-imidazolyl, 1-(3-cyanophenyl)-4-imidazolyl, 1-(4-cyanophenyl)-4-imidazolyl, 1-(3-trifluoromethylphenyl)-4-imidazolyl, 1-[3-(2-hydroxyethyl)phenyl]-4-imidazolyl, 1-[3-(1-hydroxy-1-methylethyl)phenyl]-4-imidazolyl, 1-(3-methoxyphenyl)-4-imidazolyl, 1-(2-difluoromethoxyphenyl)-4-imidazolyl, 1-(3-difluoromethoxyphenyl)-4-imidazolyl, 1-(4-difluoromethoxy-phenyl)-4-imidazolyl, 1-(2-pyridyl)-4-imidazolyl, 1-(4-benzo[b]furanyl)-4-imidazolyl, 1-(5-benzo[b]furanyl)-4-imidazolyl, 1-(7-benzo[b]furanyl)-4-imidazolyl, 1-(2-quinolyl)-4-imidazolyl, 1-(3-quinolyl)-4-imidazolyl, 1-(4-quinolyl)-4-imidazolyl, 1-(5-quinolyl)-4-imidazolyl, 1-(6-quinolyl)-4-imidazolyl, 1-(8-quinolyl)-4-imidazolyl, 1-phenyl-3-pyrazolyl, 5-phenyl-3-pyrazolyl, 1-phenyl-4-pyrazolyl, 1-(2-fluorophenyl)-3-pyrazolyl, 5-(2-fluorophenyl)-3-pyrazolyl, 5-(3-fluorophenyl)-3-pyrazolyl, 1-(3-fluorophenyl)-4-pyrazolyl, 1-(4-fluorophenyl)-3-pyrazolyl, 5-(4-fluorophenyl)-3-pyrazolyl, 5-(2-chlorophenyl)-3-pyrazolyl, 5-(3-chlorophenyl)-3-pyrazolyl, 5-(4-chlorophenyl)-3-pyrazolyl, 5-(2-difluoromethoxyphenyl)-3-pyrazolyl, 5-(3-difluoromethoxyphenyl)-3-pyrazolyl, 2-methyl-5-phenyl-3-pyrazolyl, 5-(2-pyridyl)-3-pyrazolyl, 5-(2-quinolyl)-3-pyrazolyl, 5-(3-quinolyl)-3-pyrazolyl, 4-phenyl-2-thiazolyl, 5-phenyl-2-thiazolyl, 5-(3-chlorophenyl)-2-thiazolyl, 5-(4-chlorophenyl)-2-thiazolyl, 5-(4-methoxyphenyl)-2-thiazolyl, 5-(2-pyridyl)-2-thiazolyl, 2-phenyl-4-thiazolyl, 4-phenyl-2-oxazolyl, 5-phenyl-2-oxazolyl, 4-(2-fluoromethoxyphenyl)-2-oxazolyl, 4-(3-fluoromethoxyphenyl)-2-oxazolyl, 5-phenyl-3-isoxazolyl, 3-phenyl-5-isoxazolyl, 3-(2-chlorophenyl)-5-isoxazolyl, 3-(3-chlorophenyl)-5-isoxazolyl, 3-(4-chlorophenyl)-5-isoxazolyl, 3-(2-pyridyl)-5-isoxazolyl, 2-phenyl-1,2,3-triazol-4-yl, 5-phenyl-1,2,4-thiadiazol-3-yl, 5-phenyl-1,3,4-thiadiazol-2-yl, 5-(3-chlorophenyl)-1,3,4-thiadiazol-2-yl, 5-(2-pyridyl)-1,3,4-thiadiazol-2-yl, 5-(2-ethyl-4-pyridyl)-1,3,4-thiadiazol-2-yl, 5-phenyl-2-pyridyl, 6-phenyl-3-pyridyl, 2-phenyl-4-pyridyl, 5-(2-pyridyl)-2-pyridyl, 5-benzoyl-2-pyridyl, 6-benzoyl-3-pyridyl, 5-chloro-2-pyrazinyl, 5-(2-methyl-1-propenyl)-2-pyrazinyl, 5-acetyl-2-pyrazinyl, 5-propionyl-2-pyrazinyl, 5-phenyl-2-pyrazinyl, 5-(3-hydroxyphenyl)-2-pyrazinyl, 5-(4-hydroxyphenyl)-2-pyrazinyl, 5-(1,2,4-thiadiazol-5-yl)-2-pyrazinyl, 5-(1,3,4-thiadiazol-2-yl)-2-pyrazinyl, 5-(2-pyridyl)-2-pyrazinyl, 5-(3-pyridyl)-2-pyrazinyl, 5-(5-pyrimidinyl)-2-pyrazinyl, 5-(3-quinolyl)-2-pyrazinyl, 5-benzoyl-2-pyrazinyl, 5-(2-pyridylcarbonyl)-2-pyrazinyl, 5-acetyl-2-pyrimidinyl, 5-acetyl-3-methyl-2-pyrimidinyl, 4-phenyl-2-pyrimidinyl, 5-phenyl-2-pyrimidinyl, 6-phenyl-4-pyrimidinyl, 2-phenyl-5-pyrimidinyl, 5-(2-fluorophenyl)-2-pyrimidinyl, 5-(3-fluorophenyl)-2-pyrimidinyl, 5-(4-fluorophenyl)-2-pyrmidinyl, 5-(2-chlorophenyl)-2-pyrimidinyl, 5-(3-chlorophenyl)-2-pyrimidinyl, 5-(4-chlorophenyl)-2-pyrimidinyl, 5-(2-methylphenyl)-2-pyrimidinyl, 5-(3-methylphenyl)-2-pyrimidinyl, 5-(2-fluoromethylphenyl)-2-pyrimidinyl, 5-(3-fluoromethylphenyl)-2-pyrimidinyl, 5-(2-trifluoromethylphenyl)-2-pyrimidinyl, 5-(3-trifluoromethylphenyl)-2-pyrimidinyl, 5-(4-trifluoromethylphenyl)-2-pyrimidinyl, 5-(2-hydroxymethylphenyl)-2-pyrimidinyl, 5-(3-hydroxymethylphenyl)-2-pyrimidinyl, 5-(2-hydroxyphenyl)-2-pyrimidinyl, 5-(3-hydroxyphenyl)-2-pyrimidinyl, 5-(2-methoxyphenyl)-2-pyrimidinyl, 5-(3-methoxyphenyl)-2-pyrimidinyl, 5-(4-methoxyphenyl)-2-pyrimidinyl, 5-(2-fluoromethoxyphenyl)-2-pyrimidinyl, 5-(3-fluoromethoxyphenyl)-2-pyrimidinyl, 5-(2-fluoro-5-methylphenyl)-2-pyrimidinyl, 5-(3-fluoro-5-methoxyphenyl)-2-pyrimidinyl, 6-phenyl-3-pyridazinyl, 6-phenyl-1,2,4-triazin-3-yl, 5-chloro-2-benzoxazolyl, 5-fluoro-2-benzothiazolyl, 4-methyl-2-benzothiazolyl, 2-methyl-5-benzothiazolyl, 4-methoxy-2-benzothiazolyl, 3-quinolyl, 6-quinolyl, 7-methyl-2-quinolyl, 2-methyl-6-quinolyl, 6-chloro-2-quinoxalinyl, pyrido[3,2-b]pyridin-2-yl, 7-chloropyrido[3,2-b]pyridin-2-yl, 7-methylpyrido[3,2-b]pyridin-2-yl, 7-trifluoromethylpyrido[3,2-b]pyridin-2-yl, 7-difluoromethoxypyrido[3,2-b]pyridin-2-yl, 7-acetylpyrido[3,2-b]pyridin-2-yl, and the like, preferably 3,4-dichlorophenyl, 4-acetylphenyl, 5-oxo-5,6,7,8-tetrahydro-2-naphthyl, 4-acetyl-3-trifluoromethylphenyl, 4-(1-ethyl-2-imidazolyl)phenyl, 4-benzoylphenyl, 4-(2-pyridylcarbonyl)phenyl, 1-phenyl-3-pyrrolyl, 1-phenyl-4-imidazolyl, 1-(2-fluorophenyl)-4-imidazolyl, 1-(3,5-difluorophenyl)-4-imidazolyl, 1-(3-chlorophenyl)-4-imidazolyl, 1-(3-cyanophenyl)-4-imidazolyl, 1-[3-(2-hydroxyethyl)phenyl]-4-imidazolyl, 1-(3-difluoromethoxyphenyl)-4-imidazolyl, 1-(7-benzo[b]furanyl)-4-imidazolyl, 1-(2-quinolyl)-4-imidazolyl, 1-(3-quinolyl)-4-imidazolyl, 1-phenyl-3-pyrazolyl, 5-phenyl-3-pyrazolyl, 1-phenyl-4-pyrazolyl, 1-(3-fluorophenyl)-4-pyrazolyl, 1-(4-fluorophenyl)-3-pyrazolyl, 5-(4-chlorophenyl)-3-pyrazolyl, 5-(3-quinolyl)-3-pyrazolyl, 5-phenyl-2-thiazolyl, 3-phenyl-5-isoxazolyl, 5-(2-methyl-1-propenyl)-2-pyrazinyl, 5-phenyl-2-pyrazinyl, 5-(3-hydroxyphenyl)-2-pyrazinyl, 5-(4-hydroxyphenyl)-2-pyrazinyl, 5-(2-pyridyl)-2-pyrazinyl, 5-benzoyl-2-pyrazinyl, 5-phenyl-2-pyrimidinyl, 5-(2-fluorophenyl)-2-pyrimidinyl, 5-(3-fluorophenyl)-2-pyrimidinyl, 5-(3-chlorophenyl)-2-pyrimidinyl, 5-(3-trifluoromethyl-phenyl)-2-pyrimidinyl, 5-chloro-2-benzoxazolyl, 4-methyl-2-benzothia-zolyl, 7-methyl-2-quinolyl, 7-trifluoromethylpyrido[3,2-b]pyridin-2-yl, and the like, especially 1-phenyl-3-pyrazolyl, 5-phenyl-3-pyrazolyl, 5-phenyl-2-pyrazinyl, 5-(3-hydroxyphenyl)-2-pyrazinyl, 5-(4-hydroxyphenyl)-2-pyrazinyl, 5-phenyl-2-pyrimidinyl, 5-(2-fluorophenyl)-2-pyrimidinyl, 5-(3-fluorophenyl)-2-pyrimidinyl, 7-trifluoro-methylpyrido[3,2-b]pyridin-2-yl, and the like.

n represents 0 or 1, 0 is preferable.

T, U, V and W represent independently nitrogen atom or methine which may have a substituent selected from the group consisting of halogen, lower alkyl, hydroxy and lower alkoxy, where at least two of them represent the said methine group.

“Methine which may have a substituent selected from the group consisting of halogen, lower alkyl, hydroxy and lower alkoxy” refers to unsubstituted methine or methine having a substituent which can be selected from the group consisting of halogen, lower alkyl, hydroxy and lower alkoxy.

Halogen atom as the aforesaid substituent includes preferably fluorine atom, chlorine atom, and the like.

Lower alkyl as the aforesaid substituent includes preferably methyl, ethyl, and the like.

Lower alkoxy as the aforesaid substituent includes preferably methoxy, ethoxy, and the like.

The aforesaid substituent include preferably halogen, and the like.

The preferred mode of T, U, V and W includes, for example, T, U, V and W are independently methine optionally having the aforesaid substituent, preferably halogen; or one of T, U, V and W is nitrogen atom.

X represents methine or nitrogen.

Y represents imino which may be substituted with lower alkyl, or oxygen.

“Imino which may be substituted with lower alkyl” refers to unsubstituted imino or imino substituted with lower alkyl.

The aforesaid lower alkyl includes, preferably, methyl, ethyl, and the like.

Y is preferably unsubstituted imino or oxygen, especially oxygen.

The term “pharmaceutically acceptable salts” refers to the pharmaceutically acceptable and common salts, for example, a base addition salt to carboxyl group when the compound has a carboxyl group, or an acid addition salt to amino or basic heterocyclyl when the compound has an amino or basic heterocyclyl group, including quaternary ammonium salts, prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, trifluoro acetate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to PYY, PYY₃₋₃₆, PYY agonists, and anti-obesity agents are meant to also include the pharmaceutically acceptable salts and esters thereof.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The “pharmaceutically acceptable esters” in the present invention refer to non-toxic esters, for example, the pharmaceutically acceptable, common esters on carboxyl group when the compound has a carboxyl group, for example, esters with lower alkyls (for example methyl, ethyl, propyl, isopropyl, butyl, sec-butyl, tert-butyl, pentyl, isopentyl, neopentyl, cyclopropyl, cyclobutyl, cyclopentyl), aralkyls (for example benzyl, phenethyl), lower alkenyls (for example allyl, 2-butenyl), lower alkoxy (lower) alkyls (for example methoxymethyl, 2-methoxyethyl, 2-ethoxyethyl), lower alkanoyloxy (lower) alkyls (for example acetoxymethyl, pivaloyloxy-methyl, 1-pivaloyloxyethyl), lower alkoxycarbonyl (lower) alkyls (for example methoxycarbonylmethyl, isopropoxycarbonylmethyl), carboxy-(lower)alkyls (for example carboxymethyl), lower alkoxycarbonyloxy-(lower)alkyls (for example 1-(ethoxycarbonyloxy)ethyl, 1-(cyclohexyl-oxycarbonyloxy)ethyl), carbamoyloxy(lower)alkyls (for example carbamoyloxymethyl), phthalidyl group, (5-substituted-2-oxo-1,3-dioxol-4-yl)methyl (for example (5-methyl-2-oxo-1,3-dioxol-4-yl)methyl), and the like.

The compounds in the compositions of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, or tautomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of obesity and obesity-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower-back pain, gallbladder disease, gout, kidney cancer, and increased anesthetic risk. The compositions of the present invention are also useful for reducing the risk of secondary outcomes of obesity, such as reducing the risk of left ventricular hypertrophy. The compositions of the present invention are also useful to treat Alzheimer's disease.

The term “metabolic syndroime”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIIDM, also known as Type I diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, metabolic syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds or combinations of the present invention to reduce food intake, to reduce body weight, or to maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce food intake, to reduce body weight, or to maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, metabolic syndrome, hypercholesterolemia, hypertriglyceridemia, and cholelithiasis.

The terms “administration of” and or “administering a” composition should be understood to mean providing a composition of the invention to a subject in need of treatment or prevention.

The instant compositions include administration of a single dosage formulation which contains PYY, PYY₃₋₃₆ or a PYY agonist in combination with a second anti-obesity agent, as well as administration of each of the two active agents (PYY, PYY₃₋₃₆ or PYY agonist, and an anti-obesity agent) in its own separate dosage formulation. A single dosage formulation will provide convenience for the patient, which is an important consideration especially for patients with diabetes or obese patients who may be in need of multiple medications. However, separate dosage formulations and routes of administration may be required. The present invention also includes administration of two separate dosage formulations at different times, at different dosages and in different frequencies. The separate dosage formulations may also be given at different times of the day depending on the duration of action of the individual components. Where separate dosage formulations are used, the individual components of the composition may be administered at essentially the same time, i.e., concurrently, or at separately staggered times, i.e. sequentially prior to or subsequent to the administration of the other component of the composition. Administration in these various ways are suitable for the present compositions as long as the beneficial pharmaceutical effect of the combination of PYY, PYY₃₋₃₆ or a PYY agonist, and the second anti-obesity agent is realized by the patient at substantially the same time. Such beneficial effect is preferably achieved when the target blood level concentrations of each active drug are maintained at substantially the same time. It is preferred that the combination of PYY, PYY₃₋₃₆ and the PYY agonist, and the second anti-obesity agent be co-administered concurrently on a once-a-day dosing schedule; however, varying dosing schedules, such as PYY, PYY₃₋₃₆ or the PYY agonist once, twice, three times or more per day, and the anti-obesity agent once, twice, three times or more times per day, is also encompassed herein. For example, PYY, PYY₃₋₃₆ and the PYY agonist may be administered one to six times a day within up to two hours before or after each meal, whereas the anti-obesity agent may be given at one, two or three times a day. An effective amount of PYY or an agonist thereof can be administered in a single dose, or in multiple doses, for example two to six doses daily, during a course of treatment. In another embodiment, PYY is administered whenever the effect (e.g., decreased food intake, or weight loss) is desired. In another embodiment, PYY, PYY₃₋₃₆ or the PYY agonist is administered slightly prior to whenever the effect is desired, such as, but not limited to about 10 minutes, about 15 minutes, about 30 minutes, about 45 minutes, about 60 minutes, about 90 minutes, or about 120 minutes, prior to the time the effect is desired. In another embodiment, a time release formulation is utilized. In another embodiment, a therapeutically effective amount of PYY, PYY₃₋₃₆ or a PYY agonist is administered as a single pulse dose, as a bolus dose, or as pulse doses administered over time. Thus, in pulse doses, a bolus administration of PYY₃₋₃₆ is provided, followed by a time period wherein no PYY₃₋₃₆ is administered to the subject, followed by a second bolus administration. In specific, non-limiting examples, pulse doses of PYY₃₋₃₆ are administered during the course of a day, during the course of a week, or during the course of a month.

The instant pharmaceutical composition is therefore to be understood to include all such regimes of simultaneous or alternating treatment, as well as the use of two dosage formulations that require different routes of administration, and the terms “administration” and “administering” are to be interpreted accordingly. For example, PYY, PYY₃₋₃₆ or a PYY agonist in a first unit dosage form may be administered by inhalation using a nasal spray, or by injection via a syringe; and the anti-obesity agent in the second unit dosage form may be orally administered.

The term “administration” as used herein refers to modes of parenteral and peripheral routes of administration which include intravenous, intramuscular, intraperitoneal, intrasternal, transdermal, sublingual, buccal, inhaled, subcutaneous and intrarticular injection, infusion and intranasal administration.

The term “subject”, as used herein refers to an animal, preferably a mammal, most preferably a human, who has been the object of treatment, observation or experiment. In one embodiment, the subject is acclimatized. In another embodiment the subject is not under stress.

The term “subject in need thereof” refers to a subject who is in need of treatment or prophylaxis as determined by a researcher, veterinarian, medical doctor or other clinician. In one embodiment, the subject in need of treatment is an obese mammal. In another embodiment, the subject in need of treatment is an obese human with one or more obesity-related co-morbidities. In another embodiment, the subject in need of treatment is an obese human without obesity-related co-morbidities.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of obesity or an obesity-related disorder in subjects as risk for obesity or the obesity-related disorder.

The magnitude of prophylactic or therapeutic dose of the active ingredients (e.g. PYY, PYY₃₋₃₆, PYY agonist, anti-obesity agent) of the composition will, of course, vary with the nature of the severity of the condition to be treated and with the particular compound in the composition and its route of administration. It will also vary according to the age, weight and response of the individual patient.

In general, the daily dose range of PYY, PYY₃₋₃₆ or a PYY agonist formulation for reducing food intake, reducing body weight, maintaining body weight reduction, treating obesity and/or an obesity related disorder such as diabetes mellitus and/or metabolic syndrome lies within the dosage range of from about 10 μg to about 1 g per day, preferably given in a single dose or in divided dosages one to six times a day, or in sustained release form. For example, a therapeutically effective amount of PYY, PYY3-6 or a PYY agonist can vary from about 0.01 μg/kg to about 1 g/kg of body weight, preferably about 0.01 μg/kg to about 10 mg/kg body weight, more preferably about 0.5 mg/kg to about 10 mg/kg of body weight, or most preferably about 0.5 mg/kg to about 5 mg/kg of body weight.

Alternatively, a non-limiting range for a therapeutically effective amount or dose of PYY, PYY₃₋₃₆ or a PYY agonist within the methods and formulations of the present invention is between about 0.001 pmol (picomoles)/kg to about 10 pmol/kg body weight, preferably between about 0.1 pmol/kg to about 135 pmol/kg body weight, more preferably between about 0.5 pmol/kg to about 135 pmol/kg of body weight, or about 72 pmol/kg of body weight. For all methods disclosed herein, the dose of PYY or PYY₃₋₃₆ can be based on the physiological levels observed post-prandially. The normal circulating levels of PYY₃₋₃₆ are about 8 pmol/litre (pM), typically rising to about 40 to 60 pmol/litre after a meal. The selected dose may be administered for example, by injection, including subcutaneous injection. In one embodiment, a dose of PYY or PYY₃₋₃₆ at 0.143 n moles ( 1/7th of a mole) is administered per kilogram of bodyweight, to achieve a dose that is similar to the postparandial level of PYY. Specific, non-limiting examples of doses include, but are not limited to, doses that produce the effect demonstrated when the serum levels of PYY are from about 40 pM to about 50 pM, or from about 40 pM to about 45 pM, or to about 43 pM. PYY agonists can be used at analogous doses. The dose of an agonist can be a molar equivalent of the therapeutically effective dose of PYY or PYY₃₋₃₆. However, it may be necessary to use dosages outside these limits in some cases.

For use where a PYY, PYY₃₋₃₆ or a PYY agonist formulation for nasal administration is employed, a suitable dosage range is from about 0.01 μg/kg to about 500 μg/kg, preferably from about 1 μg/kg to about 10 μg/kg of bodyweight of PYY, PYY₃₋₃₆, or a PYY agonist given in a single dose or in divided doses one to six times a day, or in sustained release form. In a standard dose, a subject will receive from about 10 μg to about 1600 μg, preferably from about 75 μg to about 800 μg, more preferably from about 100 μg to about 400 μg, most preferable between about 100 μg/kg to about 200 μg/kg. For nasal administration, a suitable intranasal dose is about 5, 10, 20, 25, 30, 40, 50, 75, 100, 150, 200, 250, 300, 350, 400, 500, 600, 700, 800, 900, 1000, 1200, 1400, 1600 μg/dose, at a dosing frequency of up to about 6 doses a day, preferably about 3 doses a day before or after every meal. Repeated intranasal dosing with the compositions of the present invention, on a schedule ranging from about 5 minutes to about 24 hours between doses, preferably between about 10 minutes to about 6 hours between doses may be administered several times a day, preferably 30 minutes before a meal or when hunger occurs.

For use where a PYY, PYY₃₋₃₆ or a PYY agonist formulation for intravenous administration is employed, a suitable dosage range is from about 0.01 μg/kg to about 1 g/kg of body weight, preferably from about 0.01 μg/kg to about 10 mg/kg, more preferably from about 0.5 mg/kg to about 10 mg/kg, most preferably from about 0.5 mg/kg to about 5 mg/kg of body weight of PYY, PYY₃₋₃₆ or a PYY agonist in the composition given in a single dose or in divided doses one to six times a day, preferably up to 3 times a day. In one embodiment, the dosage range is about 0.5 pM (picoMolar) to about 135 pM/kg of bodyweight, preferably about 72 pM/kg of body weight given in a single dose or in divided doses one to six times a day, or in sustained release form.

In general, for reducing food intake, reducing body weight, maintaining body weight reduction, treating obesity and/or an obesity related disorder such as diabetes mellitus and/or metabolic syndrome, the Mc4r agonist of the present invention, including Compound A, is administered at a daily dosage of from about 0.001 milligram to about 100 milligram per kilogram of animal body weight, preferably given in a single dose or in divided doses two to six times a day, or in sustained release form.

In general, for reducing food intake, reducing body weight, maintaining body weight reduction, treating obesity and/or an obesity related disorder such as diabetes mellitus and/or metabolic syndrome, the CB-1 antagonists/inverse agonist, including AM-251, is administered at a daily dosage of from about 0.0001 mg/kg to about 100 mg/kg of body weight orally. Generally satisfactory results may be achieved for the methods of the present invention when the CB-1 inverse agonist/antagonist, or a pharmaceutically acceptable salt or ester thereof, such as AM-251, is administered at a daily oral dosage of from about 0.001 mg/kg to about 100 mg/kg, preferably from about 0.001 mg/kg to about 10 mg/kg of body weight, given in a single dose or in divided doses two to six times a day, or in sustained release form.

In general, for reducing food intake, reducing body weight, maintaining body weight reduction, treating obesity and/or an obesity related disorder such as diabetes mellitus and/or metabolic syndrome, the NPY5 antagonist, such as Compound B, is administered at a daily dosage of from about 0.0001 mg/kg to about 100 mg/kg of body weight orally. More specifically, when treating obesity and/or obesity related disorders generally satisfactory results may be obtained when an NPY5 antagonist of Formula I, including Compound B, or a pharmaceutically acceptable salt or ester thereof, is administered at a daily oral dosage of from about 0.001 mg/kg to about 100 mg/kg, preferably from about 0.001 mg/kg to about 10 mg/kg of body weight, given in a single dose or in divided doses two to six times a day, or in sustained release form.

Celecoxib administered at a dose of about 100 mg or about 200 mg or about 400 mg, preferably oral in a single dose or in divided doses. Etoricoxib may be administered at a dose of about 60 mg, about 90 mg or about 120 mg, preferably oral in a single dose or in divided doses. Rofecoxib may be administered at a dose of about 12.5 mg or about 25 mg, preferably oral in a single dose or in divided doses. Valdecoxib administered at a dose of about 10 mg or about 20 mg, preferably oral in a single dose or in divided doses.

Leptin may be administered at a daily dosage of from about 0.01 mg/kg to about 20 mg/kg, preferably, from about 0.01 mg/kg to about 0.3 mg/kg, preferably injected in a single dose or in divided doses.

Metformin may be administered at a daily dosage of from about 0.01 mg/kg to about 100 mg/kg, preferably from about 1 mg/kg to about 50 mg/kg in a single dose or in divided doses 2 to 3 times per day, or in sustained release form; more preferably the daily dose is 500 mg, 850 mg, 1000 mg, 1500 mg, 2000 mg or 2550 mg orally given as a single dose or in divided doses 2 to 3 times per day.

Nalmefene may be administered at a daily dosage of from about 0.0001 mg/kg to about 10 mg/kg, preferably from about 0.001 to about 0.05 mg/kg.

Orlistat may be administered at a daily dosage of from about 20 mg to about 1200 mg, preferably from about 120 mg to 400 mg in a single dose or divided doses 2 to 3 times per day or in sustained release form; more preferably a 120 mg single dose 3 times per day, or in sustained release form.

Sibutramine may be administered at a daily dosage of from about 0.01 mg/kg to about 10 mg/kg, preferably from about 0.01 mg/kg to about 1 mg/kg in a single dose or in divided doses 2 to 3 times per day, or in sustained release form; more preferably the single daily dose of sibutramine is 5 mg, 10 mg, 15 mg, 20 mg or 30 mg orally.

Rimonabant may be administered at a daily dosage of from about 0.01 mg/kg to about 8 mg/kg, more preferably from about 0.3 mg/kg to about 3 mg/kg of body weight in a single dose or in divided doses 2 to 3 times per day, or in sustained release form.

Topiramate (Topamax®) may be administered at a daily dosage of from about 10 mg to about 1,600 mg per day, preferably from about 50 mg to about 400 mg per day in a single dose or in divided doses, or in sustained release form.

Zonasamide may be administered at a daily dosage of from about 10 mg to about 1,500 mg per day, preferably from about 100 mg to about 600 mg per day in a single dose or in divided doses, or in sustained release form. More preferably zonasamide may be administered at a daily dosage of from about 100 mg/d orally, with gradual increase to 400 mg/d and further increase to 600 mg/d for patients losing less than 5% of body weight at the end of 12 weeks.

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

The weight ratio of PYY, PYY₃₋₃₆ and the PYY agonist to the second anti-obesity agent may be varied and will depend upon the effective dose of each ingredient. Thus, for example, when PYY₃₋₃₆ is combined with a second anti-obesity agent, such as a NPY5 antagonist, the weight ratio of PYY₃₋₃₆ to the NPY5 antagonist will generally range from about 1000:1 to about 1:1000, preferably about 200:1 to about 1:200. Compositions of PYY, PYY₃₋₃₆ or a PPY agonist, and other anti-obesity agents will generally also be within the aforementioned range, but in each case, an effective dose of each active ingredient should be used.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing PYY, PYY₃₋₃₆ or a PYY agonist, additional active ingredient(s) such as a second anti-obesity agent, and pharmaceutically acceptable excipients.

Any suitable route of administration may be employed for providing a subject, especially a human, with an effective dosage of a composition of the present invention. For example, oral, rectal, topical, parenteral (including subcutaneous injection, intramuscular, IV infusion), ocular (opthalmic), pulmonary (aerosol inhalation), nasal administration, and the like may be employed. The route of administration may be via a single bolus delivery, via continuous delivery (e.g. continuous transdermal, mucosal, or intravenous delivery) over an extended period of time, or in a repeated administration protocol (e.g. hourly, daily, weekly, or monthly). Dosage forms include tablets, troches, dispersions, suspensions, nasal sprays, injectable solutions, solutions, capsules, creams, ointments, aerosols, and the like.

The pharmaceutical compositions of the present invention comprise a combination of PYY, PYY₃₋₃₆ or a PYY agonist, and a second anti-obesity agent, as active ingredients or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. PYY, and PYY3-6 are amphoteric, therefore, they may be utilized as free bases, and as pharmaceutically acceptable salts, including acid or metal salts. Metal salts include alkali and alkaline earth metal salts, e.g., potassium or sodium salts. In particular, the term “pharmaceutically acceptable salts” refers to salts prepared from pharmaceutically acceptable non-toxic bases or acids including inorganic bases or acids and organic bases or acids.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

For administration by inhalation, PYY, PYY3-36, PYY agonist or a composition of the present invention for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients. In the case of a pressurized aerosol the dosage unit can be determined by providing a valve to deliver a metered amount. Capsules and cartridges of e.g., gelatin for use in an inhaler or insufflator can be formulated containing a powder mix of the compound; and a suitable powder base such as lactose or starch. For inhalation, PYY, PYY3-36 or a PYY agonist is administered as an aerosol or a dispersion in a carrier. In one specific, non-limiting example, PYY, PYY3-36, or an agonist thereof is administered as an aerosol from a conventional valve, such as, but not limited to, a metered dose valve, through an aerosol adapter also known as an actuator. A suitable fluid carrier can be also included in the formulation, such as, but not limited to, air, a hydrocarbon, such as e-butane, propane, isopentane, amongst others, or a propellant, such as, but not limited to a fluorocarbon. Optionally, a stabilizer is also included, and/or porous particles for deep lung delivery are included (e.g., see U.S. Pat. No. 6,447,743). Compounds with poor solubility in aqueous systems require formulation by using solubilizing agents such as ionic surfactants, chelates, polyethylene glycol (PEG), ethanol, or other agents which may have undesirable effects when used for inhalation. In addition, a treatment requiring successful delivery into alveoli of the lower pulmonary region may preclude from the formulation the use of certain irritants such as chlorofluorocarbons and should involve a minimum number of; required doses. Alternatively, to avoid such limitations, liposomes or hydrophobic particles can be used. In one embodiment, an inhalation formulation for a sustained release includes using aerosol droplet particles approximately 1-2.1 μm in size, or less than 1 μm in size. Small particle aerosol liposomes and liposome-drug combinations for medical use have been previously described (e.g., see EP 87309854). For intranasal administration, nasal formulations can be administered using any spray bottle or syringe. Suitable nasal spray formulations of the compositions of the present invention, or of PYY, PYY 3-36, or the PYY agonist active ingredient of the present compositions, include, but are not limited to, those disclosed in US 2004/0115135 and WO 2004/056314.

PYY, PYY agonists, and PYY antagonists useful in the methods of this disclosure can be provided as parenteral compositions, e.g., for injection or infusion. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan. Preferably, they are suspended in an aqueous carrier, for example, in an isotonic i buffer solution at a pH of about 3.0 to about 8.0, preferably at a pH of about 3.5 to 10 about 7.4, 3.5 to 6.0, or 3.5 to about 5.0. Useful buffers include sodium citrate-citric acid and sodium phosphate-phosphoric acid, and sodium acetate/acetic acid buffers. For parenteral administration compositions include suitable aqueous solutions of an active ingredient in water-soluble form, for example in the form of a water-soluble salt, or aqueous injection suspensions that contain viscosity-altering substances, for example sodium carboxymethylcellulose, sorbitol and/or dextran, and, if desired, stabilizers. The active ingredient, optionally together with excipients, can also be in the form of a lyophilisate and can be made into a solution prior to parenteral administration by the addition of suitable solvents. Solutions such as those that are used, for example, for parenteral administration can also be used as infusion solutions. A form of repository or “depot” slow release preparation may be used so that therapeutically effective amounts of the preparation are delivered into the bloodstream over many hours or days following transdermal injection or delivery.

Various pharmaceutically acceptable carriers and their formulation are described in standard formulation treatises, e.g., Remington's Pharmacentical Sciences by E. W. Martin. See also Wang, Y. J. and Hanson, M. A., Journal of Parenteral Science and Technology, Technical Report 5 No. 10, Supp. 42:2 S, 1988. The formulation preferably does not include oxidizing agents and other compounds that are known to be deleterious to PPY, PYY₃₋₃₆ and PYY agonists. Generally, the formulations are prepared by contacting the PPY, PYY₃₋₃₆ and PYY agonist, uniformly and intimately with liquid carriers or finely divided solid carriers or both. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. PPY, PYY₃₋₃₆ and PYY agonists are also suitably administered by sustained-release systems.

Suitable examples of sustained-release formulations of PPY, PYY₃₋₃₆ and PYY agonists include suitable polymeric materials (such as, for example, semi-permeable polymer matrices in the Loran of shaped articles, e.g., films, or mirocapsules), suitable hydrophobic materials (for example as an emulsion in an acceptable oil) or ion exchange resins, and sparingly soluble derivatives (such as, for example, a sparingly soluble salt). Sustained-release PPY, PYY₃₋₃₆ and PYY agonist compositions may be administered orally, rectally, parenterally, intravaginally, intraperitoneally, topically (as by powders, ointments, gels, drops or transdermal patch), bucally, or as an oral or nasal spray. Sustained release matrices include polylactides (U.S. Pat. No. 3,773,919, EP 58,481), copolymers of L-glutamic acid and gamma-ethyl-L-glutamate (Sideman et al., Biopolymers 22: 547-556, 1983, poly(2-hydroxyethyl methacrylate)); (Larger et al., Biomed. Mater. Res. 15: 167-277, 1981; Langer, Chem. Tech. 12: 98-105, 1982, ethylene vinyl acetate (Larger et al., Id.) or poly-D-(−)-3-hydroxybutyric acid (EP 133,988). Sustained-release PPY, PYY₃₋₃₆ and PYY agonists include liposomal PPY and PYY agonists (see generally, Langer, Science 249: 1527-1533, 1990; Treat et al., Liposomes in the Therapy of Infectious Disease and Cancer, Lopez-Berestein and Fidler (eds.), 317-27; 353-65, (1989)). Liposomes containing PPY and PYY analogs are prepared by methods known per se: DE 3,218,121; Epstein et al., Proc. Matl. Acad. Sci. U.S.A. 82: 3688 3692, 1985; Hwang et al., Proc. Natl. Acad. Sci. U.S.A. 77: 4030-4034, 1980; EP 52,322; EP 36,676; EP 88,046; EP 143,949; EP 142,641; Japanese Patent Application No. 83-118008; U.S. Pat. No. 4,485,045, U.S. Pat. No. 4,544,545; and EP 102,324. Ordinarily, the liposomes are of the small (about 200-800 Angstroms) unilamellar type in which the lipid content is greater than about 30 mole percent cholesterol, the selected proportion being adjusted for the optimal performance. Preparations for administration can be suitably formulated to give controlled release of PYY, PYY₃₋₃₆ and PYY agonists. For example, the pharmaceutical compositions may be in the form of particles comprising a biodegradable polymer and/or a polysaccharide jellifying and/or bioadhesive polymer, an amphiphilic polymer, an agent modifying the interface properties of the particles and a pharmacologically active substance. These compositions exhibit certain biocompatibility features which allow a controlled release of the active substance. See U.S. Pat. No. 5,700,486. In yet an additional embodiment, PPY, PYY₃₋₃₆ and PYY agonists are delivered by way of a pump (see Langer, supra; Sefton, CRC Crit. Ref Biomed. Eng. 14: 201, 1987; Buchwald et al., Surgery 88: 507, 1980; Saudek et al., N. Engl. J; Med. 321: 574, 1989) or by continuous subcutaneous infusions, for example, using a mini-pump. An intravenous bag solution may also be employed. Other controlled release systems are discussed in the review by Langer (Science 249: 1527-1533, 1990). In another aspect of the disclosure, PPY, PYY₃₋₃₆ and PYY agonists are delivered by way of an implanted pump, described, for example, in U.S. Pat. No. 6,436,091; U.S. Pat. No. 5,939,380; U.S. Pat. No. 5,993,414. Implantable drug infusion devices are used to provide patients with a constant and long term dosage or infusion of a drug or any other therapeutic agent. An example of such an active drug infusion device currently available is the Medtronic SynchroMed programmable pump. An example of such a pump is described listed in U.S. Pat. No. 5,728,396.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilainellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such, polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. PYY, PYY₃₋₃₆, PYY agonist, and anti-obesity agent) can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Desirably, each tablet, cachet, capsule, or liquid formulation contains from 10 μg to about 1 g, particularly 10, 15, 20, 25, 30, 40, 50, 75, 100, 125, 150, 175, 200, 225, 250 μg; and 1, 5, 10, 15, 20, 25, 50, 75, 100, 150, 200, 250, 300, 400, 500, 750, 850 and 1,000 milligrams of PYY, PYY₃₋₃₆, or a PYY agonist, for the symptomatic adjustment of the dosage to the subject to be treated. Each tablet, cachet, capsule or liquid formulation contains from about 0.001 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of an anti-obesity agent (such as NPY5 antagonist, melanocortin 4 R agonist, CB-1 inverse agonist/antagonist) for the symptomatic adjustment of the dosage to the subject to be treated. Exemplifying the invention is a pharmaceutical composition comprising PYY, PYY₃₋₃₆, or a PYY agonist, and a second anti-obesity agent described above and a pharmaceutically acceptable carrier. Also exemplifying the invention is a pharmaceutical composition made by combining PYY, PYY₃₋₃₆, or a PYY agonist, and a second anti-obesity agent described above and a pharmaceutically acceptable carrier. An illustration of the invention is a process for making a pharmaceutical composition comprising combining any of PYY, PYY₃₋₃₆, or the PYY agonists described above, and any second anti-obesity agent described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Injectable Suspension (I.M.) | mg/mL |
|---|---|
| PYY, PYY agonist or PYY₃₋₃₆ | 1 |
| Compound A, Compound B or AM-251 | 1 |
| cyclodextrin | Q.S. ed to |
| (35% weight/volume) | 1 ml volume |
| glycerol | 63.05 |

Water for injection to a total volume of 1 mL

| Tablet | mg/tablet |
|---|---|
| PYY, PYY agonist, or PYY₃₋₃₆ | 10 |
| Compound A, Compound B or AM-251 | 20 |
| Microcrystalline Cellulose | 40.5 |
| Lactose | 114.5 |
| Croscarmellose Sodium | 5.0 |
| Hydroxypropylcellulose | 8.0 |
| Sodium Dodecyl Sulfate | 1.0 |
| Magnesium Stearate | 1.0 |
| | 200 mg |

| Capsule | mg/capsule |
|---|---|
| PYY, PYY agonist, or PYY₃₋₃₆ | 5 |
| Compound A, Compound B or AM-251 | 20 |
| Lactose | 60 |
| Sodium Dodecyl Sulfate | 15 |
| | 100 |

| Aerosol or Nasal Spray | Per canister | |
|---|---|---|
| PYY, PYY agonist, or PYY₃₋₃₆ | 15 | mg |
| Compound A, Compound B or AM-251 | 300 | mg |
| Lecithin, NF Liq. Conc. | 1.2 | mg |
| Cyclodextrin | 4.025 | g |
| Water | 12.15 | g |

It will be understood that the scope of compositions of the compounds of this invention with other agents useful for treating or preventing obesity and obesity-related conditions includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

### In vivo Study of the Effect of PYY₃₋₃₆ on 4 Hour and 16 Hour Food Intake and Body Weight Gain in DIO Mice

Materials and Methods

PYY₃₋₃₆ was obtained from PeptidoGenic Research & Co., Inc. (Livermore, Calif.), CB1 antagonist AM251 was obtained from Tocris Cookson (Ellisville, Mo.).

Mice were individually housed in micro-isolator cages (Labproducts™). The animal room was set on a 12-hour light, 12-hour dark cycle (lights on at 7:00 am), with temperature and relative humidity maintained at 23+2° C. and 55±15%, respectively. Male C57BL6 mice (Taconic Farm) were fed on a MHF diet (Research diet 12451, 45% kcal from fat) at 8 weeks of age to develop diet-induced-obesity (DIO). The DIO mice used in the experiment were of 13 months of age, and were maintained on the MHF diet for 11 months. The average body weight of the mice was 55-56 grams. DIO mice feeding ad libitum were administered intraperitonelly (IP) with saline (n=12) and human PYY₃₋₃₆ (0.03, 0.1, 0.5, 1.0, 2.0, 5.0 mg/kg, n=10 per group) before lights out. Pre-weighted diet was provided after dosing. 4-hour, 16-hour food intake (FI) and 16-hour body weight (BW) gain were measured.

Results

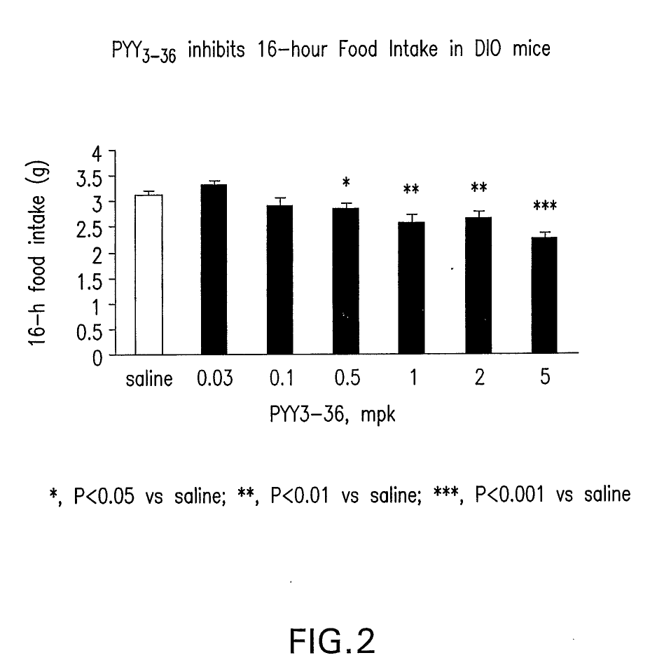

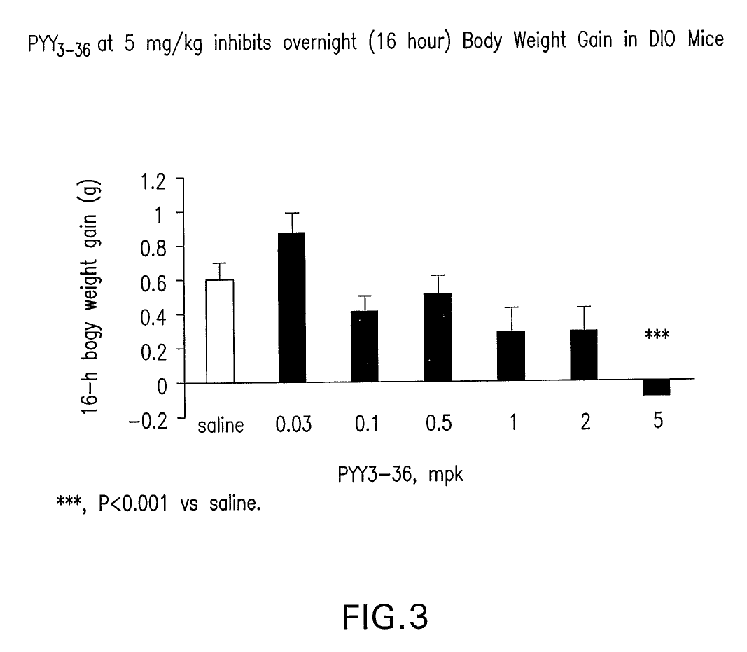

PYY₃₋₃₆ at doses greater than 0.5 mg/kg significantly inhibited 4-hour and 16-hour food intake. PYY₃₋₃₆ at 5 mg/kg significantly inhibited 16 h body weight gain. FIG. 1 and FIG. 2 show that PYY₃₋₃₆ at doses of 0.5, 1.0, 2.0, and 5 mg/kg significantly reduced acute 4-hour and 16-hour food intake. FIG. 3 shows that PYY₃₋₃₆ at 5 mg/kg significantly reduced body weight gain. This data confirms that PYY₃₋₃₆ has anorectic effects in DIO mice.

These results suggest that PYY₃₋₃₆ will be effective for the treatment of obesity.

### EXAMPLE 2

### In vivo Study of the Effect of the Combination of PYY₃₋₃₆ and CB-1 Inverse Agonist AM-251, on Food Intake and Bodyweight

Materials and Methods

PYY₃₋₃₆ was obtained from PeptidoGenic Research & Co., Inc. (Livermore, Calif.), CB1 antagonist AM251 was obtained from Tocris Cookson (Ellisville, Mo.).

Male C57BL6 mice (Taconic Farm) were fed on a MHF diet (Research diet 12451, 45% kcal from fat) at 8 weeks of age to develop diet-induced-obesity (DIO). The DIO mice used in the experiment were of 10 months of age, and were maintained on the MHF diet for 8 months. The average body weight of the mice was 57-58 grams. DIO mice feeding ad libitum on the MHF diet were conditioned by oral gavage (PO) with vehicle (5% Tween80 and 0.5% methylcellulose) and IP injection with saline. The mice were randomized and divided into 4 groups, which were administered with: vehicle; PYY₃₋₃₆ (1 mg/kg, IP); AM251 (1 mg/kg, PO); and PYY₃₋₃₆ (1 mg/kg, IP)+AM251 (1 mg/kg, PO). Sample sizes were n=12 for the vehicle group, and n=10 for the drug groups. The compound administration was performed one hour before the dark cycle following measurement of body weight.

Results

This study shows that the combination of PYY₃₋₃₆ and AM251 had greater anorectic effect on 4-hour food intake than either PYY₃₋₃₆ or AM251 given alone (FIG. 4). PYY₃₋₃₆ and AM251 co-administration also exhibited a greater reduction in 16 hour food intake than PYY₃₋₃₆ alone.

These results suggest that the combination of PYY₃₋₃₆ and a CB-1 inverse agonist will be more effective for the treatment of obesity than monotherapy with either compound alone.

### EXAMPLE 3

### In vivo Study of the Effect of the Combination of PYY₃₋₃₆ and Melanocortin 4 Receptor Agonist, Compound A, on Food Intake

Materials and Methods

PYY₃₋₃₆ was obtained from PeptidoGenic Research & Co., Inc. (Livermore, Calif.), Compound A was synthesized at Merck Research Laboratories.

Male C57BL6 mice (Taconic Farm) were fed on a MHF diet (Research diet 12451, 45% kcal from fat) at 8 weeks of age to develop diet-induced-obesity (DIO). The DIO mice used in the experiment were of 10 months of age, and were maintained on the MHF diet for 8 months. The average body weight of the mice was 57-58 grams. DIO mice feeding ad libitum were conditioned by oral gavage (PO) with vehicle (5% Tween80 and 0.5% methylcellulose) and intraperitonel (IP) injection with saline. The mice were randomized and divided into 4 groups, which were administered with: vehicle; PYY₃₋₃₆ (1 mg/kg, IP); Compound A (20 mg/kg, PO); and PYY₃₋₃₆ (1 mg/kg, IP)+Compound A (20 mg/kg, PO). Sample sizes were n=12 for the vehicle group, and n=10 for the drug groups. The compound administration was performed one hour before the dark cycle following measurement of body weight.

Results

The maximum inhibition of food intake was observed with the combination of PYY₃₋₃₆ and Compound A administration (FIG. 6).

These results suggest that the combination of PYY₃₋₃₆ and a melanocortin 4 receptor agonist will be more effective for the treatment of obesity than monotherapy with either compound alone.

Effective anti-obesity combinations result in a greater body weight change when PYY₃₋₃₆ (or PYY or a PYY agonist) and the second anti-obesity agent are given together, than the body weight change seen with either compound is administered alone.

### EXAMPLE 4

### In vivo Study of the Effect of the Combination of PYY₃₋₃₆ and Neuropeptide Y5 Antagonist, Compound B, on Food Intake and Body Weight

Materials and Methods

PYY₃₋₃₆ and compound B were synthesized at Banyu Pharmaceutical Co., Ltd. Male lean and DIO C57BL/6 mice (CLEA Japan Inc., Tokyo, Japan) were used. DIO mice (17 months old at the experiment) were prepared by feeding a moderately high-fat (MHF) diet (Oriental BioService Kanto Inc., Ibaraki, Japan) for over a year before the start of drug administration. The average body weight of the mice was 57 grams; (n=12). DIO mice feeding ad libitum were conditioned by oral gavage (PO) with vehicle (5% Tween80 and 0.5% methylcellulose) and intraperitonel (IP) injection with saline. The mice were randomized and divided into 4 groups, which were administered with: vehicle; PYY₃₋₃₆ (1 mg/kg, SC); Compound B (30 mg/kg, oral); and PYY₃₋₃₆ (1 mg/kg, SC)+Compound B (30 mg/kg, oral). The compound administration was performed one hour before the dark cycle following measurement of body weight. The mice were monitored for 2 weeks.

Results

The maximum inhibition of food intake (FIG. 9) and decrease in bodyweight (FIG. 10) was observed with the combination of PYY₃₋₃₆ and Compound B administration. The body composition of the mice, as determined by NMR, showed a greater reduction in fat mass for the combination treatment than for each compound alone. These results suggest that the combination of PYY₃₋₃₆ and a neuropeptide Y5 receptor antagonist will be more effective for the treatment of obesity than monotherapy with either compound alone.

Effective anti-obesity combinations result in a greater decrease in food intake and body weight when PYY₃₋₃₆ (or PYY or a PYY agonist) and the second anti-obesity agent are given together, than the food intake decrease and body weight decrease seen with either compound is administered alone.

### EXAMPLE 5

### Human Study for Combination Therapy with a PYY₃₋₃₆ and a Second Anti-Obesity Agent, such as NPY5 Antagonist, Compound B

Materials and Methods

800 people with a BMI>30 are advised to diet and increase their physical activity. After a two-week placebo run-in period, which includes a standardized program of diet, physical activity, and lifestyle changes, the patients are randomized into 4 treatment groups: 1) placebo; 1) an effective dose of PYY₃₋₃₆, such as 200 μg/dose given 30 minutes prior to each meal; 3) an effective dose of a second anti-obesity agent, such as 1 mg of NPY5 antagonist, Compound B, given once a day; and 4) an effective dose of PYY₃₋₃₆ plus an effective dose of the NPY5 antagonist, Compound B. PYY₃₋₃₆ is administered intranasally 45 minutes before each meal as previously determined to be effective. The NPY5 antagonist is given in tablet form at once per day, as previously determined to be effective. Patients are treated for 6 months, body weights are measured weekly, and appetite, hunger, satiety are measured weekly using standard questionnaires.

Effective anti-obesity combinations result in a greater body weight change when PYY₃₋₃₆ and the second anti-obesity agent, such as the NPY5 antagonist, are given together, than the body weight change seen with either compound is administered alone.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-47. (canceled)

48. A composition comprising
(a) PYY, PYY₃₋₃₆, or a PYY agonist, or a pharmaceutically acceptable salt or ester thereof; and
(b) an anti-obesity agent selected from the group consisting of:
(1) 5HT transporter inhibitor;
(2) NE transporter inhibitor;
(3) ghrelin antagonist;
(4) H3 antagonist/inverse agonist;
(5) MCH1R antagonist;
(6) MCH2R agonist/antagonist;
(7) MC3R agonist;
(8) NPY1 antagonist;
(9) NPY4 agonist;
(10) NPY5 antagonist;
(11) leptin;
(12) leptin agonist/modulator;
(13) leptin derivatives;
(14) opioid antagonist;
(15) orexin antagonist;
(16) BRS3 agonist;
(17) 11β HSD-1 inhibitor;
(18) CCK-A agonist;
(19) CNTF;
(20) CNTF agonist/modulator;
(21) CNTF derivative;
(22) Cox-2 inhibitor;
(23) GHS agonist;
(24) 5HT2C agonist;
(25) 5HT6 antagonist;
(26) monoamine reuptake inhibitor;
(27) UCP-1, 2, and 3 activator;
(28) β3 agonist;
(29) thyroid hormone β agonist;
(30) PDE inhibitor;
(31) FAS inhibitor;
(32) DGAT1 inhibitor;
(33) DGAT2 inhibitor;
(34) ACC2 inhibitor;
(35) glucocorticoid antagonist;
(36) acyl-estrogens;
(37) lipase inhibitor;
(38) fatty acid transporter inhibitor;
(39) dicarboxylate transporter inhibitor;
(40) glucose transporter inhibitor;
(41) serotonin reuptake inhibitor;
(42) a minorex;
(43) amphechloral;
(44) amphetamine;
(45) axokine;
(46) benzphetamine;
(47) chlorphentermine;
(48) clobenzorex;
(49) cloforex;
(50) clominorex;
(51) clortermine;
(52) cyclexedrine;
(53) dextroamphetamine;
(54) diphemethoxidine,
(55) N-ethylamphetamine;
(56) fenbutrazate;
(57) fenisorex;
(58) fenproporex;
(59) fludorex;
(60) fluminorex;
(61) furfurylmethylamphetamine;
(62) levamfetamine;
(63) levophacetoperane;
(64) mefenorex;
(65) metamfepramone;
(66) methamphetamine;
(67) norpseudoephedrine;
(68) pentorex;
(69) phendimetrazine;
(70) phenmetrazine;
(71) phytopharm 57;
(72) picilorex;
(73) topiramate; and
(74) zonisamide;
and pharmaceutically acceptable salts and esters thereof.

49. The composition of claim 48 wherein the anti-obesity agent selected from the group consisting of:
(a) PYY, PYY₃₋₃₆, or a PYY agonist, or a pharmaceutically acceptable salt or ester thereof; and
(b) an anti-obesity agent selected from the group consisting of:
(1) 5HT transporter inhibitor;
(2) NE transporter inhibitor;
(3) ghrelin antagonist;
(4) H3 antagonist/inverse agonist;
(5) MCH1R antagonist;
(6) MCH2R agonist/antagonist;
(7) MC3R agonist;
(8) NPY1 antagonist;
(9) NPY4 agonist;
(10) NPY5 antagonist;
(11) leptin;
(12) leptin agonist/modulator;
(13) leptin derivatives;
(14) opioid antagonist;
(15) orexin antagonist;
(16) BRS3 agonist;
(17) 11β HSD-1 inhibitor;
(18) CCK-A agonist;
(19) CNTF;
(20) CNTF agonist/modulator;
(21) CNTF derivative;
(22) Cox-2 inhibitor;
(23) GHS agonist;
(24) 5HT2C agonist;
(25) 5HT6 antagonist;
(26) monoamine reuptake inhibitor;
(27) UCP-1, 2, and 3 activator;
(28) β3 agonist;
(29) thyroid hormone β agonist;
(30) PDE inhibitor;
(31) FAS inhibitor;
(32) DGAT1 inhibitor;
(33) DGAT2 inhibitor;
(34) ACC2 inhibitor;
(35) glucocorticoid antagonist;
(36) acyl-estrogens;
(37) lipase inhibitor;
(38) fatty acid transporter inhibitor;
(39) dicarboxylate transporter inhibitor;
(40) glucose transporter inhibitor; and
(41) serotonin reuptake inhibitor;
and pharmaceutically acceptable salts and esters thereof.

50. The composition of claim 48 wherein the anti-obesity agent selected from the group consisting of:
(1) NE transporter inhibitor;
(2) ghrelin antagonist;
(3) H3 antagonist/inverse agonist;
(4) MCH1R antagonist;
(5) MCH2R agonist/antagonist;
(6) MC3R agonist;
(7) NPY1 antagonist;
(8) NPY4 agonist;
(9) NPY5 antagonist;
(10) orexin antagonist;
(11) BRS3 agonist;
(12) 11β HSD-1 inhibitor;
(13) CNTF;
(14) CNTF agonist/modulator;
(15) CNTF derivative;
(16) Cox-2 inhibitor;
(17) GHS agonist;
(18) monoamine reuptake inhibitor;
(19) UCP-1, 2, and 3 activator;
(20) thyroid hormone β agonist;
(21) PDE inhibitor;
(22) FAS inhibitor;
(23) DGAT1 inhibitor;
(24) DGAT2 inhibitor;
(25) ACC2 inhibitor;
(26) glucocorticoid antagonist;
(27) acyl-estrogens;
(28) lipase inhibitor;
(29) fatty acid transporter inhibitor; and
(30) dicarboxylate transporter inhibitor;
and pharmaceutically acceptable salts and esters thereof.

51. The composition of claim 48 wherein the anti-obesity agent is selected from the group consisting of:
(1) acyl-estrogen;
(2) opioid antagonist;
(3) monoamine reuptake inhibitor;
(4) lipase inhibitor;
(5) leptin;
(6) CNTF;
(7) CNTF derivatives; and
(8) NPY5 antagonist;
and pharmaceutically acceptable salts and esters thereof.

52. The composition of claim 48 wherein the acyl-estrogen is selected from oleoyl-estrone, or a pharmaceutically acceptable salt or ester thereof.

53. The composition of claim 48 wherein the monoamine reuptake inhibitor is selected from sibutramine, or a pharmaceutically acceptable salt or ester thereof.

54. The composition of claim 48 wherein the CNTF derivative is selected from axokine, or a the pharmaceutically acceptable salt or ester thereof.

55. The composition of claim 48 wherein the lipase inhibitor is selected from orlistat, or a pharmaceutically acceptable salt or ester thereof.

56. The composition of claim 48 wherein the anti-obesity agent is selected from leptin, or a pharmaceutically acceptable salts or ester thereof.

57. The composition of claim 48 wherein the opioid antagonist is selected from nalmefene, or a pharmaceutically acceptable salt or ester thereof.

58. The composition of claim 48 wherein the anti-obesity agent selected from the group consisting of a NPY5 antagonist, or a pharmaceutically acceptable salt or ester thereof.

59. The composition of claim 58 wherein the NPY5 antagonist is selected from the group consisting of a compound of formula I: 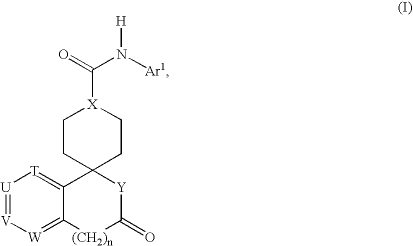
and pharmaceutically acceptable salts and esters thereof, wherein
Ar¹ is selected from the group consisting of:
(1) aryl, and
(2) heteroaryl,
wherein the aryl and heteroaryl groups are unsubstituted or optionally substituted with a substituent selected from the group consisting of:
(a) halogen,
(b) nitro,
(c) lower alkyl,
(d) halo(lower)alkyl,
(e) hydroxy(lower)alkyl,
(f) cyclo(lower)alkyl,
(g) lower alkenyl,
(h) lower alkoxy,
(i) halo(lower)alkoxy,
(j) lower alkylthio,
(k) carboxyl,
(l) lower alkanoyl,
(m) lower alkoxycarbonyl,
(n) lower alkylene optionally substituted with oxo, and
(o) -Q-Ar²;
Ar² is selected from the group consisting of
(1) aryl, and
(2) heteroaryl,
wherein aryl and heteroaryl are unsubstituted or optionally substituted with a substituent selected from the group consisting of:
(a) halogen,
(b) cyano,
(c) lower alkyl,
(d) halo(lower)alkyl,
(e) hydroxy(lower)alkyl,
(f) hydroxy,
(g) lower alkoxy,
(h) halo(lower)alkoxy,
(i) lower alkylamino,
(j) di-lower alkylamino,
(k) lower alkanoyl, and
(l) aryl;
n is 0 or 1;
Q is selected from the group consisting of a single bond or carbonyl;
T, U, V and W are each independently selected from the group consisting of
(1) nitrogen, and
(2) methine,
wherein the methine group is unsubstituted or optionally substituted with a substituent selected from the group consisting of
(a) halogen,
(b) lower alkyl,
(c) hydroxy, and
(d) lower alkoxy; and
wherein at least two of T, U, V, and W are methine;
X is selected from the group consisting of
(1) nitrogen, and
(2) methine; and
Y is selected from the group consisting of
(1) imino, unsubstituted or optionally substituted with lower alkyl, and
(2) oxygen.

60. The composition of claim 59 wherein the anti-obesity agent is a NPY5 antagonist is selected from the group consisting of:
(1) 3-oxo-N-(5-phenyl-2-pyrazinyl)-spiro[isobenzofuran-1(3H), 4′-piperidine]-1′-carboxamide;
(2) 3-oxo-N-(7-trifluoromethylpyrido[3,2-b]pyridin-2-yl)spiro-[isobenzofuran-1(3H), 4′-piperidine]-1′-carboxamide;
(3) N-[5-(3-fluorophenyl)-2-pyrimidinyl]-3-oxospiro-[isobenzofuran-1(3H), 4′-piperidine]-1′-carboxamide;
(4) trans-3′-oxo-N-(5-phenyl-2-pyrimidinyl)spiro[cyclohexane-1,1′(3′H)-isobenzofuran]-4-carboxamide;
(5) trans-3′-oxo-N-[1-(3-quinolyl)-4-imidazolyl]spiro[cyclohexane-1,1′(3′H)-isobenzofuran]-4-carboxamide;
(6) trans-3-oxo-N-(5-phenyl-2-pyrazinyl)spiro[4-azaiso-benzofuran-1(3H), 1′-cyclohexane]-4′-carboxamide;
(8) trans-N-[5-(3-fluorophenyl)-2-pyrimidinyl]-3-oxospiro[5-azaisobenzofuran-1(3H), 1′-cyclohexane]-4′-carboxamide;
(9) trans-N-[5-(2-fluorophenyl)-2-pyrimidinyl]-3-oxospiro[5-azaisobenzofuran-1(3H), 1′-cyclohexane]-4′-carboxamide;
(10) trans-N-[1-(3,5-difluorophenyl)-4-imidazolyl]-3-oxospiro[7-azaisobenzofuran-1(3H), 1′-cyclohexane]-4′-carboxamide;
(11) trans-3-oxo-N-(1-phenyl-4-pyrazolyl)spiro[4-azaisobenzofuran-1(3H), 1′-cyclohexane]-4′-carboxamide;
(12) trans-N-[1-(2-fluorophenyl)-3-pyrazolyl]-3-oxospiro[6-azaisobenzofuran-1(3H), 1′-cyclohexane]-4′-carboxamide;
(13) trans-3-oxo-N-(1-phenyl-3-pyrazolyl)spiro[6-azaisobenzofuran-1(3H), 1′-cyclohexane]-4′-carboxamide; and
(14) trans-3-oxo-N-(2-phenyl-1,2,3-triazol-4-yl)spiro[6-azaisobenzofuran-1(3H), 1′-cyclohexane]-4′-carboxamide;
and pharmaceutically acceptable salts and esters thereof.

61. The composition of claim 48 comprising PYY, or a pharmaceutically acceptable salt or ester thereof; and an anti-obesity agent, or a pharmaceutically acceptable salt or ester thereof.

62. The composition of claim 48 comprising PYY₃₋₃₆, or a pharmaceutically acceptable salt or ester thereof; and an anti-obesity agent, or a pharmaceutically acceptable salt or ester thereof.

63. The composition of claim 48 comprising a PYY agonist, or a pharmaceutically acceptable salt or ester thereof; and an anti-obesity agent, or a pharmaceutically acceptable salt or ester thereof.

64. A composition according to claim 48 further comprising a pharmaceutically acceptable carrier.

65. The composition of claim 48 wherein the anti-obesity agent selected from the group consisting of:
(1) aminorex;
(2) amphechloral;
(3) amphetamine;
(4) benzphetamine;
(5) chlorphentermine;
(6) clobenzorex;
(7) cloforex;
(8) clominorex;
(9) clortermine;
(10) cyclexedrine;
(11) dextroamphetamine;
(12) diphemethoxidine,
(13) N-ethylamphetamine;
(14) fenbutrazate;
(15) fenisorex;
(16) fenproporex;
(17) fludorex;
(18) fluminorex;
(19) furfurylmethylamphetamine;
(20) levamfetamine;
(21) levophacetoperane;
(22) mefenorex;
(23) metamfepramone;
(24) methamphetamine;
(25) norpseudoephedrine;
(26) pentorex;
(27) phendimetrazine;
(28) phenmetrazine;
(29) phytopharm 57;
(30) picilorex;
(31) topiramate; and
(32) zonisamide;
and pharmaceutically acceptable salts and esters thereof.

66. A composition comprising PYY, PYY3-36 or a PYY agonist, or a salt or ester thereof; and an anti-obesity agent, wherein the anti-obesity agent is a CB-1 antagonist/inverse agonist selected from the group consisting of:
(1) AM 251; and
(2) rimonabant;
and pharmaceutically acceptable salts and esters thereof.

67. A composition comprising PYY, PYY3-36, or a PYY agonist, or a salt or ester thereof; and an anti-obesity agent, wherein the anti-obesity agent is a Mc4r agonist is selected from the group consisting of:
(1) 2-[2-(1-{[(3S,4R)-1-tert-butyl-4-(2,4-difluorophenyl)pyrrolidin-3-yl]carbonyl}piperidin-4-yl)-5-chloro phenyl]-N-methylcarboxamide;
(2) 2-[2-(1-{[(3S,4R)-1-tert-butyl-4-(2,4-difluorophenyl)pyrrolidin-3-yl]carbonyl}piperidin-4-yl)-5-fluoro-phenyl]-N-methylcarboxamide;
(3) 2-[2-(1-{[(3S,4R)-1-tert-butyl-4-(2,4-difluorophenyl)pyrrolidin-3-yl]carbonyl}piperidin-4-yl)-5-methyl-phenyl]-N-methylcarboxamide;
(4) 2-[2-(1-{[(3S,4R)-1-tert-butyl-4-(2,4-difluorophenyl)pyrrolidin-3-yl]carbonyl}piperidin-4-yl)-phenyl]-N-methylcarboxamide;
(5) 2-[2-(1-{[(3S,4R)-1-tert-butyl-4-(2,4-difluorophenyl)pyrrolidin-3-yl]carbonyl}piperidin-4-yl)-4-methyl-phenyl]-N-methylcarboxamide;
(6) 2-[2-(1-{[(3S,4R)-1-tert-butyl-4-(2,4-difluorophenyl)pyrrolidin-3-yl]carbonyl}piperidin-4-yl)-4-fluoro-phenyl]-N-methylcarboxamide;
(7) 4-[2-(2-azetidin-1-yl-1(S)-methyl-2-oxoethyl)-4-chlorophenyl]-1-{[(3S,4R)-1-tert-butyl-4-(2,4-difluorophenyl)pyrrolidin-3-yl]carbonyl}piperidine;
(8) 4-[2-(2-azetidin-1-yl-1(R)-methyl-2-oxoethyl)-4-chlorophenyl]-1-{[(3S,4R)-1-tert-butyl-4-(2,4-difluorophenyl)pyrrolidin-3-yl]carbonyl}piperidine;
(9) 4-[2-(2-azetidin-1-yl-1,1-dimethyl-2-oxoethyl)-4-chlorophenyl]-1-{[(3S,4R)-1-tert-butyl-4-(2,4-difluorophenyl)pyrrolidin-3-yl]carbonyl}piperidine;
(10) 4-[2-(2-azetidin-1-yl-1-cyclopropyl-2-oxoethyl)-4-chlorophenyl]-1-{[(3S,4R)-1-tert-butyl-4-(2,4-difluorophenyl)pyrrolidin-3-yl]carbonyl}piperidine;
(11) 4-[2-(2-azetidin-1-yl-1,1-dimethyl-2-oxoethyl)-4-fluorophenyl]-1-{[(3S,4R)-1-tert-butyl-4-(2,4-difluorophenyl)pyrrolidin-3-yl]carbonyl}piperidine;
(12) 4-[2-(2-azetidin-1-yl-1-cyclopropyl-2-oxoethyl)-4-fluorophenyl]-1-{[(3S,4R)-1-tert-butyl-4-(2,4-difluorophenyl)pyrrolidin-3-yl]carbonyl}piperidine;
(13) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(14) N-{(1R)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}acetamide;
(15) N-{1-[2-(1-{[(3S,4R)-1-tert-butyl-4-(2,4-difluorophenyl)pyrrolidin-3-yl]carbonyl}piperidin-4-yl)-5-chlorophenyl]-1-methylethyl}acetamide;
(16) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}acetamide;
(17) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}cyclobutanecarboxamide;
(18) N-{1-[2-(1-{[(3S,4R)-1-tert-butyl-4-(2,4-difluorophenyl)pyrrolidin-3-yl]carbonyl}piperidin-4-yl)-5-chlorophenyl]ethyl}propanamide;
(19) N-{1-[2-(1-{[(3S,4R)-1-tert-butyl-4-(2,4-difluoro-phenyl)pyrrolidin-3-yl]carbonyl}piperidin-4-yl)-5-chlorophenyl]ethyl}-N-methylurea;
(20) methyl-2-[2-(1-{[(3S,4R)-1-tert-butyl-4-(2,4-difluorophenyl)-pyrrolidin-3-yl]carbonyl}piperidin-4-yl)-5-chlorophenyl]-2-methylpropanoate;
(21) N-{1-[2-(1-{[(3S,4R)-1-tert-butyl-4-(2,4-difluoro-phenyl)pyrrolidin-3-yl]carbonyl}piperidin-4-yl)-5-fluorophenyl]-1-methylethyl}acetamide;
(22) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}-N-methylurea;
(23) N-{1-[2-(1-{[(3S,4R)-1-tert-butyl-4-(2,4-difluorophenyl)pyrrolidin-3-yl]carbonyl}piperidin-4-yl)-5-fluorophenyl]ethyl}cyclobutanecarboxamide;
(24) N-{1-[2-(1-{[(3S,4R)-1-tert-butyl-4-(2,4-difluoro-phenyl)pyrrolidin-3-yl]carbonyl}piperidin-4-yl)-5-fluorophenyl]ethyl}propanamide;
(25) N-{(1S)-1-[2-(1-{[(3S,4R)-1-tert-butyl-4-(2,4-difluorophenyl)-pyrrolidin-3-yl]carbonyl}piperidin-4-yl)-5-fluorophenyl]ethyl}acetamide;
(26) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]propyl}acetamide; and
(27) N-{(1S)-1-[2-(1-{[(3S,4R)-1-tert-butyl-4-(2,4-difluorophenyl)-pyrrolidin-3-yl]carbonyl}piperidin-4-yl)-5-chlorophenyl]ethyl}pyrimidine-5-carboxamide;
and pharmaceutically acceptable salts and esters thereof.

68. A method of treating a subject having a disorder associated with excessive food intake comprising administration of a composition according to claim 48, or a pharmaceutically acceptable salt thereof, to a subject in need of such treatment.

69. The method of claim 68 wherein the disorder associated with excessive food intake is obesity.

70. A method of treating a subject having a disorder associated with excessive food intake comprising administration of a composition according to claim 48, or a pharmaceutically acceptable salt thereof, to a subject in need of such treatment.

71. The method of claim 70 wherein the disorder associated with excessive food intake is obesity.


SEQUENCE LISTING (2 sequences)

SEQ ID NO: 1 - protein, 36 aa, Human Peptide YY
YPIKPEAPGEDASPEELNRYYASLRHYLNLVTRQRY

SEQ ID NO: 2 - protein, 34 aa, Human Peptide YY 3-36
IKPEAPGEDASPEELNRYYASLRHYLNLVTRQRY